(19) United States
(12) Patent Application Publication
(10) Pub. No.: US 2003/0220278 A1
(43) Pub. Date: Nov. 27, 2003

(54) Method of inducing proliferation of T cells

(75) Inventors: Philip J.S. Stork, Portland, OR (US); Andrey S. Shaw, St. Louis, MO (US)

Correspondence Address: KLARQUIST SPARKMAN, LLP, 121 SW SALMON STREET, SUITE 1600, PORTLAND, OR, 97204, US

(73) Assignee: Oregon Health & Science University

(21) Appl. No.: 10/350,547

(22) Filed: Jan. 23, 2003

Related U.S. Application Data
(60) Provisional application No. 60/352,309, filed on Jan. 24, 2002.
(60) Provisional application No. 60/422,341, filed on Oct. 29, 2002.

(51) Int. Cl. A61K 48/00; A61K 39/395; C12N 15/85

(52) U.S. Cl. 514/44; 435/455; 424/144.1

(57) ABSTRACT

Methods are provided to induce a T cell to proliferate. In one embodiment, the method includes introducing a therapeutically effective amount of a nucleic acid encoding B-Raf, or a functional variant of B-Raf, operably linked to a promoter, into the anergized T cell. In another embodiment, the method includes providing the T cell with a therapeutically effective amount of a composition that inhibits the expression or activity of Rap-1; thereby inducing the T cell to proliferate in response to the antigen. A method is also provided to induce a response against an antigen in a subject. In yet another embodiment, a method is provided for screening for an agent that activates B-raf. In addition, a transgenic mouse is provided. A nucleated cell of the transgenic mouse comprises a transgene encoding a B-Raf polypeptide. T cells isolated from the transgenic non-human animal have increased extracellular signal-regulated kinase activity as compared to a wild-type mouse.

### PRIORITY CLAIM

This application claims the benefit of U.S. Provisional Application No. 60/352,309, filed Jan. 24, 2002, and U.S. Provisional Application No. 60/422,341, filed Oct. 29, 2002, which are both incorporated herein by reference in their entireties.

### STATEMENT OF GOVERNMENT SUPPORT

This invention was made with United States government support from the National Institutes of Health, the United States government has certain rights in the invention.

### FIELD

This application relates to the field of immunology, specifically to the stimulation of anergic T cells.

### BACKGROUND

The immune system is the major surveillance for detecting disease states as a result of pathogenic invasion, cellular aberration as involved with neoplasia and psoriatic lesions, or other foreign bodies. The T cell can act directly to protect a mammalian host, but may also cooperate with other cells such as B cells, to further enhance protective mechanisms. The T cell has a number of mechanisms available to it that result in cellular destruction of a target, and, therefore, numerous mechanisms have evolved to prevent the T cell from attacking the host.

The thymus has evolved mechanisms whereby T cells which might attack native tissue are depleted so that only T cells capable of attacking other than native tissue are allowed to mature. The process of clonal deletion has been found to have a small but significant incidence of failure, as is evidenced by numerous autoimmune diseases, such as diabetes, lupus, rheumatoid arthritis, myasthenia gravis, multiple sclerosis, and the like. T cells are also active in recognizing allogeneic tissue and attacking such tissue, which is a problem in the case of transplantation.

Besides the mechanism of clonal deletion, there is believed to be a further mechanism for diminishing specific T cell activity, referred to as anergy. It has been suggested that T cell stimulation requires co-stimulation and that in the absence of co-stimulation, antigen presentation can result in anergy, where the T cell becomes tolerized to the antigen and is not stimulated upon a subsequent encounter with the antigen.

The induction of a T cell response requires that at least two signals be delivered by ligands on a stimulator cell to the T cell through cell surface receptors on the T cell. A primary activation signal is delivered to the T cell through the antigen-specific TcR. Physiologically, this signal is triggered by an antigen-MHC molecule complex on the stimulator cell, although it can also be triggered by other means such as phorbol ester treatment or crosslinking of the TcR complex with antibodies, e.g. with anti-CD3. To induce T cell activation, a second signal, called a costimulatory signal, is required by stimulation of the T cell through another cell surface molecule, such as CD28 or CTLA4. A CD28 molecule expressed on a T cell interacts with a B7 ligand, which is expressed on the surface of an antigen presenting cell. Thus, the minimal molecules on a stimulator cell required for T cell activation are an MHC molecule associated with a peptide antigen, to trigger a primary activation signal in a T cell, and a costimulatory molecule to trigger a costimulatory signal in the T cell. Engagement of the antigen-specific TcR in the absence of triggering of a costimulatory signal can prevent activation of the T cell and, in addition, can induce a state of unresponsiveness or anergy in the T cells.

Tumor cells express tumor antigens on their surface that can be recognized by T cells. However, it is well documented that a variety of tumors are immunogenic but do not stimulate an effective anti-tumor response in vivo. T cells normally recognize tumor antigens without B7 costimulation. These T cells activated through their T cell receptors in the absence of costimulation by B7 become anergic. Thus, there is need for methods that activate anergic T cells to respond to antigens, such to a methods to activate a T-lymphocyte response against a tumor antigen.

### SUMMARY

A method is provided to inducing a T cell to proliferate. The method includes introducing a therapeutically effective amount of a nucleic acid encoding B-Raf, or a functional variant of B-Raf, operably linked to a promoter, into the anergized T cell.

In another embodiment, a method is provided for inducing an T cell to proliferate in response to an antigen. The method includes providing the T cell with a therapeutically effective amount of a composition that inhibits the expression or activity of Rap-1; thereby inducing the T cell to proliferate in response to the antigen.

In a further embodiment, a method is provided to induce a response against an antigen in a subject. The method includes administering a therapeutically effective amount of a nucleic acid sequence including a promoter operably linked to a nucleic acid sequence encoding B-Raf, thereby inducing the immune response against the antigen.

In yet another embodiment, a method is provided for screening for an agent that activates B-raf. The method includes contacting an anergized T cell with the agent; and detecting proliferation of the T cell, thereby determining if the agent activates B-raf.

In one embodiment, a transgenic mouse is provided wherein a nucleated cell of the transgenic mouse comprises a transgene encoding a B-Raf polypeptide. T cells isolated from the transgenic non-human animal have increased extracellular signal-regulated kinase activity as compared to a wild-type mouse.

The foregoing and other features and advantages will become more apparent from the following detailed description of several embodiments, which proceeds with reference to the accompanying figures.

### BRIEF DESCRIPTION OF THE FIGURES

FIG. 1 is a bar graph demonstrating that expression of B-Raf but not Raf-1 allows Rap1 to activate Elk-1. In all conditions, Jurkat cells were transfected with both Gal4-Elk 1 and 5×Gal4-E1b/luciferase as the reporter constructs. Jurkat cells were transfected with constitutively active Rap1 (RapV12) or empty vector (pcDNA3) and B-Raf or Raf-1. Elk-1 activation was measured as luciferase activity ±SEM.

FIG. 2 is a set of panels documenting Expression of RapV12 and B-Raf transgenes in murine T lymphocytes. FIG. 2A is a digital image of a Western blot demonstrating Myc-RapV12 expression in transgenic splenocytes. Splenocytes were harvested from PCR positive (+) and negative (−) transgenic animals, the prepared lysates separated by SDS-PAGE and the proteins visualized by Western blot. Expression of endogenous Rap1 was detected in all lanes (lower band, arrow). In lymphocytes isolated form PCR-positive animals, expression of the myc-tagged RapV12 protein was detected as the higher molecular weight band. FIG. 2B is a digital image of a Western blot for B-Raf expression in lymphocytes isolated from the thymus and lymph nodes taken from wild type mice (wt) and B-Raf transgenic (tr) mice. Lysates were separated by SDS-PAGE and the proteins visualized by Western blot. Lymphocytes isolated from the lymph nodes and thymus of transgenic mice show expression of B-Raf protein at 95 kDa. Endogenous expression of B-Raf in PC12 cells was used as a positive control (PC12). FIG. 2C is a digital image of histological sections demonstrating B-Raf expression in the thymus, spleen and lymph nodes isolated from wild type (wild type; left panels) and B-Raf transgenic (B-Raf; right panels) mice. Spleen and lymph nodes were harvested from B-Raf-transgenic and wild type mice were fixed and analyzed for B-Raf expression by immunohistochemistry on 20 μm frozen sections, as indicated. B-Raf expression was only detected in the lymphoid organs from B-Raf transgenic mice. FIG. 2D is a plot of a fluorescence activated cells sorting (FACS) experiment documenting B-Raf expression in thymocytes isolated from wild type (dark gray line) and B-Raf transgenic (black line) mice was compared to wild type and B-Raf thymocytes stained with a control antibody (gray shading and broken gray line, respectively). The staining is representative of two experiments.

FIG. 3 is a series of 5 panels demonstrating transgenic expression of B-Raf increases ERK activation and T cell proliferation. FIG. 3A is a digital image of a Western blot assay. T cells were isolated from the spleens of wild type and B-Raf transgenic mice. T cells were stimulated with anti-CD3 antibody (CD3) with or without anti-CD28 antibody (CD28), and cross-linking secondary antibody for 5 minutes or left untreated (U) as indicated. Phorbol myristate acetate (PMA) was used as a positive control. T cells incubated with secondary antibody alone (2°) served as a negative control. FIG. 3B is a digital image of a Western blot. T cells were isolated from the spleen of wild type and B-Raf transgenic mice. T cells were stimulated with anti-CD3 antibody (α-CD3), and cross-linking secondary antibody for the times indicated. PMA was used as a positive control. For both FIG. 3A and FIG. 3B, lysates were prepared and the proteins separated by SDS-PAGE and visualized by Western blot. In the Western blot, phospho-ERK (pERK1/2) was measured using a phospho-specific ERK antibody. The positions of pERK1 and pERK2 are indicated, in a representative Western blot (n=3). FIG. 3C is a digital image of a Western blot. T cells isolated from wild type and transgenic mice (wild type, B-Raf, RapV12 and B-Raf×RapV12) were stimulated with increasing concentrations of anti-CD3 antibody (α-CD3), and cross-linking secondary antibody for 5 minutes. Lysates were prepared and the proteins separated by SDS-PAGE and visualized by Western blot. In the Western blot, phospho-ERK (pERK1/2) was measured using a phospho-specific ERK antibody. The positions of pERK1 and pERK2 are indicated, in a representative Western blot (n=3). FIG. 3D is a graph demonstrating T cell proliferation. Equal numbers of splenocytes from wild type (open circle) and transgenic mice [B-Raf (closed circle), RapV12 (open square), B-Raf×RapV12 (closed square)], were incubated in the presence of increasing concentrations of soluble anti-CD3 antibody (α-CD3). Data represent ³H-thymidine incorporation in counts per minute (c.p.m.) ±SEM, from a representative experiment (n=3). FIG. 3E is a bar graph of T cell proliferation. Splenocytes from wild type (wt) and B-Raf transgenic (B-Raf) animals were incubated in the presence of soluble anti-CD3 antibody (0.25 μg/ml) with or without anti-CD28 antibody (α-CD28, 2 μg/ml). Data represent ³H-thymidine incorporation in counts per minute (c.p.m.) ±SEM, (n=3).

FIG. 4 is a set of images demonstrating Rap1 and ERK are activation in anergic T cells. FIG. 4A are plots generated from a FACS analysis of CD4 and CD8 expression on thymocytes isolated from AD10 and AD10×B-Raf and AND×B-Raf animals. Thymocytes were stained with FITC-labeled anti-CD28 and Cy-Chrome-labeled anti CD4 antibodies. Viable cells were gated using forward and side scatter, and gated cells were analyzed for the expression of CD4 and CD8. FIG. 4B is a digital image of a Western blot. Anergic and non-anergic T cells from AD10 TCR transgenic mice were stimulated with anti-CD3 antibody (Barberis et al., J. Biol. Chem. 275:36532-36540, 2000) and/or anti-CD8 antibody (Lee et al., J. Exp. Med. 190:1263-1274, 1999), and cross-linking secondary antibody for 5 minutes or left untreated (UT) as indicated (see FIG. 4B rows A and D). T cell lysates were prepared and assayed for Rap1 activation using Gst-RalGDS and Western blotting was performed using polyclonal Rap1 antibody. The position of Rap1-GTP following isolation of Gst-RalGDS-bound proteins is shown (see FIG. 4B, rows B and E). Lysates were prepared and assayed for Ras activation using Gst-Raf-1-RBD and Western blotting was performed using Ras antibody. The position of Ras-GTP following isolation of Gst-Raf-1RBD-bound proteins is shown (see FIG. 4B, row C and F). T cell lysates were also prepared and assayed for ERK activation. In the Western blot, phospho-ERK (pERK1/2) was measured using a phospho-specific ERK antibody. The positions of pERK1 and pERK2 are indicated, in a representative Western blot (n=3). FIG. 4C is a digital image of a Western blot. Anergic and non-anergic T cells from AD10×B-Raf animals were stimulated with anti-CD3 antibody (Barberis et al., J. Biol. Chem. 275:36532-36540, 2000) and/or anti-CD28 antibody (Lee et al., J. Exp. Med. 190:1263-1274, 1999), and cross-linking secondary antibody for 5 minutes or left untreated (UT) as indicated (see FIG. 4C, rows A and D) T cell lysates were prepared and assayed for Rap1 activation using Gst-Ral-GDS and Western blotting was performed using polyclonal Rap1 antibody. The position of Rap1-GTP following isolation of Gst-RalGDS-bound proteins is shown (see FIG. 4C, rows B and E). Lysates were prepared and assayed for Ras activation using Gst-Raf-1-RBD and Western blotting was performed using Ras antibody. The position of Ras-GTP following isolation of Gst-Raf-1RBD-bound proteins is shown (see FIG. 4C, rows C and F). T cell lysates were also prepared and assayed for ERK activation. In the Western blot, phospho-ERK (pERK1/2) was measured using a phospho-specific ERK antibody. The positions of pERK1 and pERK2 are shown, in a representative Western blot (n=3).

FIG. 5 is a set of images demonstrating ERK activation in anergic cells is functional. FIG. 5A is a digital image of a Western Blot. T cells from non-anergic AD10 and anergic AD10×B-Raf cultures were stimulated with anti-CD3 antibody (α-CD3), for the times indicated (min). PMA was used as a positive control. Lysates were prepared and the proteins separated by SDS-PAGE and visualized by Western blot. In the Western blot, phospho-ERK (pERK1/2) was measured using a phospho-specific ERK antibody. The positions of pERK1 and pERK2 are indicated, in a representative Western blot (n=3). FIG. 5B is graph of a FACS analysis. Purified AD10 and AD10×B-Raf T cell blasts were stimulated for 18 h with plate-bound anti-CD3 and anti-CD28 antibodies. Cell surface expression of CD69 on unstimulated (gray shading) and stimulated (black line) non-anergic and anergic T cells was compared to the background staining obtained with an isotype control APC-labeled mouse IgG (gray line). The staining was representative of three experiments. FIG. 5C is a bar graph of the data from FIG. 5B presented as mean fluorescent intensity of CD69 surface staining. FIG. 5D is a digital image of a Western Blot. Non-anergic and anergic AD10 and AD10×B-Raf T cells were stimulated (st) for 3 hours with plate-bound anti-CD3 and anti-CD28 antibodies or left unstimulated (un). Nuclear extracts were prepared and for c-Fos expression was measured by Western blot. The position of c-Fos is shown in a representative Western blot (n=3).

FIG. 6 is a set of graphs showing ERK activation is not sufficient to rescue anergy. FIG. 6A is a graph of T cell proliferation. Anergic and non-anergic T cells from AD10 and AD 10×B-Raf animals were incubated with irradiated APCs in the presence of increasing concentrations of PCC peptide. Non-anergic AD10 cells (open circle), anergic AD10 cells (closed circle), non-anergic AD10×B-Raf cells (open square) and anergic AD10×B-Raf cells (closed square). Data represent ³H-thymidine incorporation in counts per minute (c.p.m) ±SEM. FIG. 6B is a graph of T cell proliferation. Anergic and non-anergic T cells from AD10 mice were incubated with irradiated APCs in the presence of increasing concentrations of PCC peptide. Non-anergic AD10 cells (open circle), with IL-2 (closed circle), anergic AD10 cells (open square), with IL-2 (closed square). Data represent ³H -thymidine incorporation in counts per minute (c.p.m) ±SEM. FIG. 6C is a graph of T cell proliferation. Anergic and non-anergic T cells from AD10×B-Raf mice were incubated with irradiated APCs in the presence of increasing concentrations of PCC peptide. Non-anergic AD10×B-Raf cells (open circle), with IL-2 (closed circle), anergic AD10×B-Raf cells (open square), with IL-2 (closed square). Data represent ³H-thymidine incorporation in counts per minute (c.p.m) ±SEM.

FIG. 7 is two digital images demonstrating that the JNK pathway is downregulated in anergic T cells. FIG. 7A is a digital image of a Western blot. Non-anergic (NA) and anergic (A) T cells from AD10 and AD10×B-Raf animals were stimulated for 1 hour (St) with anti-CD3 and anti-CD28 antibodies or left unstimulated (Un). UV light exposure was used as a positive control (+UV). FIG. 7B is a digital image of a Western blot. Non-anergic (NA) and anergic (A) T cells were stimulated with anti-CD3/anti-CD28 for the times indicated. For both (a) and (b), lysates were prepared and the proteins separated by SDS-PAGE and visualized by Western blot. Phospho-JNK (pJNK1/2) protein levels were measured using a phospho-specific JNK antibody and the positions of pJNK1 and pJNK2 indicated (upper panels), in representative Western blots (n=3). Total JNK1/2 protein levels are shown in the lower panels.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO:1 is the amino acid sequence of a B-Raf polypeptide.

SEQ ID NO:2 is a nucleic acid sequence of a nucleic acid molecule encoding B-Raf.

SEQ ID NO:3 is the nucleic acid sequence of an antisense nucleic acid that targets Rap-1.

### DETAILED DESCRIPTION

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Anergy: A state of inactivation marked by the inability of a cell, such as a T cell, to proliferate. Clonal anergy is a state of inactivation of a clonal population of T cells. Without being bound by theory, whether clonal anergy or clonal expansion occurs is determined by the absence or presence of a co-stimulatory signal (via B7). Thus, if a resting Th cell receives a T cell receptor mediated signal in the absence of the co-stimulatory signal, then the Th cell will become anergic.

Animal: Living multi-cellular vertebrate organisms, a category that includes, for example, mammals and birds. The term mammal includes both human and non-human mammals. Similarly, the term “subject” includes both human and veterinary subjects.

Antigen: A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. The term “antigen” includes all related antigenic epitopes. A “tumor antigen” is an antigen expressed on a neoplasm. Specific, non-limiting examples of tumor antigens include carcinoembryonic antigen, prostate specific antigen, a melanoma specific antigen.

Antisense, Sense, and Antigene: Double-stranded DNA (dsDNA) has two strands, a 5′->3′ strand, referred to as the plus strand, and a 3′->5′ strand (the reverse compliment), referred to as the minus strand. Because RNA polymerase adds nucleic acids in a 5′->3′ direction, the minus strand of the DNA serves as the template for the RNA during transcription. Thus, the RNA formed will have a sequence complementary to the minus strand and identical to the plus strand (except that U is substituted for T).

Antisense molecules are molecules that are specifically hybridizable or specifically complementary to either RNA or the plus strand of DNA. Sense molecules are molecules that are specifically hybridizable or specifically complementary to the minus strand of DNA. Antigene molecules are either antisense or sense molecules directed to a dsDNA target. Ribozymes are catalytic nucleic acids that specifically bind a target sequence and degrade the target.

CD3: A complex of at least five membrane bound polypeptides in mature T-lymphocytes that are non-covalently associated with one another and with the T cell receptor. The CD3 complex includes gamma, delta, epsilon, zeta, and eta subunits. When antigen binds to the T cell receptor, the CD3 complex transduces the activating signals to the cytoplasm of the T cell.

CD4: Cluster of differentiation factor 4, a T-cell surface protein that mediates interaction with the MHC class II molecule. This cell surface antigen is also known as T4, Leu-3, OKT4 or L3T4. CD4 is a 55 kDa transmembrane glycoprotein belonging to the immunoglobulin superfamily. In humans CD4 is expressed of peripheral T-cells, thymocytes, on macrophages and granulocytes. It is expressed also in a developmentally regulated manner in specific regions of the brain. The human CD4 gene is on chromosome 12pter-p12. CD4 also serves as the primary receptor site for HIV on T-cells during HIV infection.

CD4+ T cell mediated immunity: An immune response implemented by presentation of antigens to CD4+ T cells.

CD8+ T cell mediated immunity: An immune response implemented by presentation of antigens to CD8+ T cells.

CD28: This cell surface antigen is known also as T90/44 antigen or Tp44. The CD28 gene contains 4 exons and maps to human 2q33-q34. CD28 is a 44 kDa homodimeric highly glycosylated protein which is expressed on most human T-cells. CD28 is a receptor for costimulatory proteins acting on T-cells.

cDNA (complementary DNA): A piece of DNA lacking internal, non-coding segments (introns) and regulatory sequences that determine transcription. cDNA is synthesized in the laboratory by reverse transcription from messenger RNA extracted from cells.

Cytokine: Proteins made by cells that affect the behavior of other cells, such as lymphocytes. In one embodiment, a cytokine is a chemokine, a molecule that affects cellular trafficking.

Encode: A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced therefrom.

Functional fragments and variants of a polypeptide: Includes those fragments and variants that maintain one or more functions of the parent polypeptide. It is recognized that the gene or cDNA encoding a polypeptide can be considerably mutated without materially altering one or more the polypeptide's functions. First, the genetic code is well-known to be degenerate, and thus different codons encode the same amino acids. Second, even where an amino acid substitution is introduced, the mutation can be conservative and have no material impact on the essential functions of a protein (see Stryer, Biochemistry 3rd Ed., 1988). Third, part of a polypeptide chain can be deleted without impairing or eliminating all of its functions. Fourth, insertions or additions can be made in the polypeptide chain—for example, adding epitope tags—without impairing or eliminating its functions (Ausubel et al., 1997).

Other modifications that can be made without materially impairing one or more functions of a polypeptide include, for example, in vivo or in vitro chemical and biochemical modifications or which incorporate unusual amino acids. Such modifications include, for example, acetylation, carboxylation, phosphorylation, glycosylation, ubiquination, labeling, e.g., with radionuclides, and various enzymatic modifications, as will be readily appreciated by those well skilled in the art. A variety of methods for labeling polypeptides and of substituents or labels useful for such purposes are well known in the art, and include radioactive isotopes such as ³²P, ligands which bind to labeled antiligands (e.g., antibodies), fluorophores, chemiluminescent agents, enzymes, and antiligands. Functional fragments and variants can be of varying length. For example, some fragments have at least 10, 25, 50, 75, 100, or 200 amino acid residues.

In one embodiment, a functional fragment or variant of B-Raf includes a fragment or variant that can induce an anergized T cell to proliferate.

Interleukin-2 (IL-2): IL2 is a protein of 133 amino acids (15.4 kDa) with a slightly basic pI. It does not display sequence homology to any other factors, although murine and human IL-2 display a homology of approximately 65 percent.

IL-2 is synthesized as a precursor protein of 153 amino acids with the first 20 aminoterminal amino acids functioning as a hydrophobic secretory signal sequence. The human gene encoding IL-2 contains four exons. The human IL-2 gene maps to human chromosome 4q26-28 (murine chromosome 3). Under physiological conditions IL-2 is produced mainly by CD4+ T-cells following their activation by mitogens or allogens. Resting cells do not produce IL2, although transformed T-cells and B-cells, leukemia cells, and NK-cells secrete IL-2.

Immune response: A response of a cell of the immune system, such as a B cell, T cell to a stimulus. In one embodiment, the response is specific for a particular antigen (an “antigen-specific response”). In one embodiment, an immune response is a T cell response, such as a Th1, Th2, or Th3 response.

Inflammation: When damage to tissue occurs, the body's response to the damage is usually inflammation. The damage may be due to trauma, lack of blood supply, hemorrhage, autoimmune attack, transplanted exogenous tissue or infection. This generalized response by the body includes the release of many components of the immune system (e.g. IL-1 and TNF), attraction of cells to the site of the damage, swelling of tissue due to the release of fluid and other processes.

Interleukin-2 (IL-2): IL-2 is produced mainly by T-cells expressing the surface antigen CD4 following their activation by mitogens or allogens. Several secondary signals are required for maximal expression of IL-2.

IL2 is a protein of 133 amino acids (15.4 kDa) with a slightly basic pI. It does not display sequence homology to any other factors. Murine and human IL-2 display a homology of approximately 65 percent. IL-2 is synthesized as a precursor protein of 153 amino acids with the first 20 aminoterminal amino acids functioning as a hydrophobic secretory signal sequence. The protein contains a single disulfide bond (positions Cys58/105) essential for biological activity.

The human IL-2 gene contains four exons. The IL-2 gene maps to human chromosome 4q26-28 (murine chromosome 3). Translocations, deletions, and/or chromosomal gene amplifications of the IL2 gene have been observed neither under physiological nor under pathological conditions. The homology of murine and human IL2 is 72 percent at the nucleotide level in the coding region.

The synthesis of IL-2 is regulated at the level of transcription. T-cells contain a labile repressor that modulates the post-transcriptional processing of IL-2 mRNA precursors. This repressor prevents post-transcriptional processing so that approximately 98 percent of the IL-2 mRNA remain unprocessed.

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

Mammal: This term includes both human and non-human mammals. Similarly, the term “subject” includes both human and veterinary subjects.

Mitogen-activated protein kinases (MPAKs): Kinases that are regulated by tyrosine and threonine phosphorylation via phosphorylation by MEKs (MAP kinase kinase). In mammalian cells there are two well characterized and highly related classical MAPKs termed Extracellular signal Regulated Kinases (ERK), named ERK-1 and ERK-2. A number of less related protein kinases have been identified including ERK3, ERK5 and ERK6. The activating phosyphorylation site for MAPKs is the amino acid sequence Thr-Glu-Tyr

The ERKs are the prototypic MAPKs and are activated by a variety of mitogenic stimuli as well as differentiation signals. MAPK activation largely requires Ras activation although activation of protein kinase C operates in a Ras-independent manner. In Drosophila, the ERK homologue is encoded by the Rolled locus (an activated allele) which has been placed downstream of Ras in the sevenless receptor kinase pathway. One of its bonafide targets in fruit flies is Drosophila Jun. ERKs are inactivated by dephosphorylation by specific protein phosphatases such as MKP1 (CL100) and PAC1. Downstream substrates include Elk1, phospholipase A2 and p90Rsk1 (another protein kinase).

MAP kinase kinase (MEK): A dual-specificity kinase that phosphorylates the tyrosine and threonine residues on ERKs 1 and 2 for activation. Two related genes encode MEK1 and MEK2, which differ in their binding to ERKs and, possibly, in their activation profiles.

MEKs are substrates for several protein kinases including the Rafs (c-, A- and B-), Mos, Tpl-2, and MEKK1 (MAP kinase kinase kinase). MEKs are phosphorylated by these kinases at two serine residues (218 and 222 in rat MEK1).

Nucleic acid: A deoxyribonucleotide or ribonucleotide polymer in either single or double stranded form, and unless otherwise limited, encompasses known analogues of natural nucleotides that hybridize to nucleic acids in a manner similar to naturally occurring nucleotides.

Oligonucleotide: A linear polynucleotide sequence of up to about 200 nucleotide bases in length, for example a polynucleotide (such as DNA or RNA) which is at least 6 nucleotides, for example at least 15, 50, 100 or even 200 nucleotides long.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Pharmaceutical agent or drug: A chemical compound or composition capable of inducing a desired therapeutic or prophylactic effect when properly administered to a subject.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polynucleotide: A linear nucleotide sequence, including sequences of greater than 100 nucleotide bases in length.

Polypeptide: Any chain of amino acids, regardless of length or post-translational modification (e.g., glycosylation or phosphorylation).

Preventing or treating a disease: “Preventing” a disease refers to inhibiting the full development of a disease, for example in a person who is known to have a predisposition to a disease such as an autoimmune disorder. An example of a person with a known predisposition is someone with a history of diabetes in the family, or who has been exposed to factors that predispose the subject to a condition, such as lupus or rheumatoid arthritis. “Treatment” refers to a therapeutic intervention that ameliorates a sign or symptom of a disease or pathological condition after it has begun to develop.

Portion of a nucleic acid sequence: At least 10, 20, 30 or 40 contiguous nucleotides of the relevant sequence, such as a sequence encoding an antigen. In some instances it would be advantageous to use a portion consisting of 50 or more nucleotides. For instance, when describing a portion of an antigen (such as an antigenic epitope), it may be advantageous to remove a portion of the relevant sequence comprising at least 10, 20, 30, 40 or 50 nucleotides up to a length.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. The promoter can be a constitutive or an inducible promoter. In one embodiment, a promoter is a T cell specific promoter, which directs transcription in T cells such as CD4+ T cells.

Purified: The term purified does not require absolute purity; rather, it is intended as a relative term. Thus, for example, a purified peptide preparation is one in which the peptide or protein is more enriched than the peptide or protein is in its natural environment within a cell. Preferably, a preparation is purified such that the protein or peptide represents at least 50% of the total peptide or protein content of the preparation.

Raf: A 74 kDa serine/threonine kinase that is activated in vivo. Inactive Raf is brought to the membrane by active GTP-Ras. Raf then is activated and phosphorylates MEK, a known in vivo target of Raf (Morrison and Cutler, Curr. Opin. Cell. Biol. 9(2):174-179, 1997). At the membrane, RasGTP has some effect on Raf kinase activity through the Raf zinc finger (Roy et al., J. Biol. Chem. 272(32):20139-20145, 1997).

Raf has essentially two domains, an N-terminal negative regulatory domain and a C-terminal catalytic domain (Cutler et al., Proc Natl Acad Sci USA 95(16):9214-9219, 1998). There are at least three isoforms, A-, B- and C-Raf. Each of the isoforms varies in its basal kinase activity and ability to be activated by different molecules (Marais et al., J. Biol. Chem. 272(7):4378-4383, 1997). B-Raf is the predominant form of Raf in neural cells, which is activated by cAMP, directly by protein kinase A phosphorylation and by Rap binding (Vossler et al., Cell 89(1):73-82, 1997). In the cytosol, inactive Raf is in a complex with 14-3-3 proteins and some heat shock proteins. Protein kinase C phosporylates Raf (Marais et al., Science 280(5360):109-112, 1998), as does protein kinase A, Pak3, and Src (Kikuchi and Williams, J. Biol Chem 271(1):588-94, 1996; King et al., Nature 396(6707):180-18, 1998; Stokoe and McCormick, EMBO J. 16(9):2384-2396 1997).

Rap-1: A member of the Ras superfamily that is an antagonist of Ras dependent transformation of fibroblasts. Rap1 inhibits ERK activation by blocking Ras-dependent activation of the MAP kinase Raf-1. Rap1 is constitutively active in anergic T cells.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. Similarly, a recombinant protein is one encoded for by a recombinant nucleic acid molecule.

T Cell: A white blood cell critical to the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8 T cells is a cytotoxic T lymphocytes. In another embodiment, a CD8 cell is a suppressor T cell.

Therapeutically effective dose: A dose sufficient to prevent advancement, or to cause regression of the disease, or which is capable of relieving symptoms caused by the disease, such as pain or swelling.

Tolerance: Diminished or absent capacity to make a specific immune response to an antigen. Tolerance is often produced as a result of contact with an antigen under non-immunizing conditions. In one embodiment, a B cell response is reduced or does not occur. In another embodiment, a T cell response is reduced or does not occur. Alternatively, both a T cell and a B cell response can be reduced or not occur.

Transduced and Transfected: A virus or vector “transduces” a cell when it transfers nucleic acid into the cell. A cell is “transfected” by a nucleic acid transduced into the cell when the DNA becomes stably replicated by the cell, either by incorporation of the nucleic acid into the cellular genome, or by episomal replication. As used herein, the term transfection encompasses all techniques by which a nucleic acid molecule might be, introduced into such a cell, including transfection with viral vectors, transformation with plasmid vectors, and introduction of naked DNA by electroporation, lipofection, and particle gun acceleration.

Vector: A nucleic acid molecule as introduced into a host cell, thereby producing a transformed host cell. A vector may include nucleic acid sequences that permit it to replicate in the host cell, such as an origin of replication. A vector may also include one or more selectable marker genes and other genetic elements known in the art. The term “vector” includes viral vectors, such as adenoviruses, adeno-associated viruses, vaccinia, and retroviruses vectors.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in practice or testing, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

The MAP kinase cascade governs multiple cellular processes in T cell function, including proliferation and differentiation. Activation of the MAP kinase ERK (extracellular signal-regulated kinase) is required for proliferation of T cells via engagement of the T cell receptor (TCR). Additionally, ERK signaling in peripheral T cells is important for IL-2-dependent proliferation (Whitehurst et al., J. Immunol. 156:1020-1029, 1996). However, ERK activation can cause growth arrest in genetic models of T cell function (Chen et al., J. Immunol:163:5796-5805, 1999) and during T cell restimulation (Lee et al., J. Exp. Med. 190:1263-1274, 1999). ERK activation dictates positive selection in the thymus (Alberola-Ila et al., Nature 373:620-623, 1995; Werlen et al., Nature 406:422-426, 2000) and may also play a role in negative selection as well (Bommhardt et al., J. Immunol. 163:715-722, 1999). The differentiation of CD4⁺/CD8⁺ T cells into the CD4 lineage also depends on ERK activation (Sharp et al., Immunity 7:609-618, 1997; Zhou et al., “CD5 costimulation up-regulates the signaling to extracellular signal-regulated kinase activation in CD4+ CD8+ thymocytes and supports their differentiation to the CD4 lineage,” J. Immunol. 164:1260-1268, 2000), with activation of ERKs favoring differentiation into the CD4 lineage and inhibition of ERKs favoring differentiation into the CD8 lineage. In addition, the magnitude of ERK activation may regulate the response of T cells to TCR engagement (Chen et al., J. Immunol. 163:5796-5805, 1999; van Den Brink et al., J Immunol. 164:469-480, 2000). Not surprisingly, the level of ERK activation in T cells is tightly regulated to control both the magnitude and duration of ERK activation.

One mechanism of ERK regulation utilizes the small G protein, Rap1 . Rap1, a member of the Ras super-family, was identified as an antagonist of Ras dependent transformation in fibroblasts (Kitayama et al., Cell 56:77-84, 1989). In many cell types, including T cells, Rap1 has been shown to inhibit ERK activation by blocking Ras-dependent activation of the MAP kinase kinase, Raf-1 (Carey et al., Mol. Cell. Biol. 20:8409-8419, 2000; Schmitt et al., J. Biol. Chem. 275:25342-25350, 2000; Xing et al., Mol. Cell. Biol. 20:7363-7377, 2000). Rap1 is activated upon TCR engagement (Carey et al., Mol. Cell. BioL 20:8409-8419, 2000; Kitayama et al., Cell 56:77-84, 1989) and can limit TCR-induced signals to ERK (Carey et al., Mol. Cell. Biol. 20:8409-8419, 2000). Rap1 activation may also provide regulation of ERK signaling in other situations. In models of anergy, elicited by tonic stimulation through the TCR alone, Rap1 has been proposed to be constitutively active and limits signaling downstream of Ras (Boussiotis et al., Science 278:124-128, 1997).

Previously was difficult to study the physiological function of modest changes in ERK activation in T cells. As disclosed herein, the introduction of B-Raf into T cells activates ERK, and induces proliferation of anergized T Cells.

### Use of B-Raf to Induce Proliferation of T Cells

A method is provided herein to induce a T cell to proliferate, such as an anergized T cell, by administering to the T cell a therapeutically effective amount of a nucleic acid encoding B-Raf, or a functional variant of B-Raf, operably linked to a promoter. In one embodiment, the T cell is in vivo. In another embodiment, the T cell is in vitro. In yet a further embodiment, The T cell expresses CD4 and/or CD28.

Nucleic acids encoding B-Raf are disclosed in GenBank Accession No. M95712, which is herein incorporated by reference in its entirety. Moreover, an amino acid sequence of a human B-Raf polypeptide (SEQ ID NO:1), and the nucleotide sequence of a mRNA encoding human B-Raf polypeptide (SEQ ID NO:2), are shown below. An amino acid sequence of a B-Raf polypeptide:

(SEQ ID NO:1)

MAALSGGGGGGAEPGQALFNGDMEPEAGAGRPAASSAADPAIPEEVWNIK
QMIKLTQEHIEALLDKFGGEHNPPSIYLEAYEEYTSKLDALQQREQQLLE
SLGNGTDFSVSSSASMDTVTSSSSSSLSVLPSSLSVFQNPTDVARSNPKS
PQKPIVRVFLPNKQRTVVPARCGVTVRDSLKKALMMRGLIPECCAVYRIQ
DGEKKPIGWDTDISWLTGEELHVEVLENVPLTTHNFVRKTFFTLAFCDFC
RKLLFQGFRCQTCGYKFHQRCSTEVPLMCVNYDQLDLLFVSKFFEHHPIP
QEEASLAETALTSGSSPSAPASDSIGPQILTSPSPSKSIPIPQPFRPADE
DHRNQFGQRDRSSSAPNVHINTIEPVNIDDLIRDQGFRGDGGSTTGLSAT
PPASLPGSLTNVKALQKSPGPQRERKSSSSSEDRNRMKTLGRRDSSDDWE
IPDGQITVGQRIGSGSFGTVYKGKWHGDVAVKMLNVTAPTPQQLQAFKNE
VGVLRKTRHVNILLFMGYSTKPQLAIVTQWCEGSSLYHHLHIIETKFEMI
KLIDIARQTAQGMDYLHAKSIIHRDLKSNNIFLHEDLTVKIGDFGLATVK
SRWSGSHQFEQLSGSILWMAPEVIRMQDKNPYSFQSDVYAFGIVLYELMT
GQLPYSNINNRDQIIFMVGRGYLSPDLSKVRSNCPKAMKRLMAECLKKKR
DERPLFPQILASIELLARSLPKIHRSASEPSLNRAGFQTEDFSLYACASP
KTPIQAGGYGAFPVH

A sequence of a mRNA encoding B-Raf:

(SEQ ID NO:2)

CGCCTCCCGG CCCCCTCCCC GCCCGACAGC GGCCGCTCGG
GCCCCGGCTC TCGGTTATAA GATGGCGGCG CTGAGCGGTG
GCGGTGGTGG CGGCGCGGAG CCGGGCCAGG CTCTGTTCAA
CGGGGACATG GAGCCCGAGG CCGGCGCCGG CCGGCCCGCG
GCCTCTTCGG CTGCGGACCC TGCCATTCCG GAGGAGGTGT
GGAATATCAA ACAAATGATT AAGTTGACAC AGGAACATAT
AGAGGCCCTA TTGGACAAAT TTGGTGGGGA GCATAATCCA
CCATCAATAT ATCTGGAGGC CTATGAAGAA TACACCAGCA
AGCTAGATGC ACTCCAACAA AGAGAACAAC AGTTATTGGA
ATCTCTGGGG AACGGAACTG ATTTTTCTGT TTCTAGCTCT
GCATCAATGG ATACCGTTAC ATCTTCTTCC TCTTCTAGCC
TTTCAGTGCT ACCTTCATCT CTTTCAGTTT TTCAAAATCC
CACAGATGTG GCACGGAGCA ACCCCAAGTC ACCACAAAAA
CCTATCGTTA GAGTCTTCCT GCCCAACAAA CAGAGGACAG
TGGTACCTGC AAGGTGTGGA GTTACAGTCC GAGACAGTCT
AAAGAAAGCA CTGATGATGA GAGGTCTAAT CCCAGAGTGC
TGTGCTGTTT ACAGAATTCA GGATGGAGAG AAGAAACCAA
TTGGTTGGGA CACTGATATT TCCTGGCTTA CTGGAGAAGA
ATTGCATGTG GAAGTGTTGG AGAATGTTCC ACTTACAACA
CACAACTTTG TACGAAAAAC GTTTTTCACC TTAGCATTTT
GTGACTTTTG TCGAAAGCTG CTTTTCCAGG GTTTCCGCTG
TCAAACATGT GGTTATAAAT TTCACCAGCG TTGTAGTACA
GAAGTTCCAC TGATGTGTGT TAATTATGAC CAACTTGATT
TGCTGTTTGT CTCCAAGTTC TTTGAACACC ACCCAATACC
ACAGGAAGAG GCGTCCTTAG CAGAGACTGC CCTAACATCT
GGATCATCCC CTTCCGCACC CGCCTCGGAC TCTATTGGGC
CCCAAATTCT CACCAGTCCG TCTCCTTCAA AATCCATTCC
AATTCCACAG CCCTTCCGAC CAGCAGATGA AGATCATCGA
AATCAATTTG GGCAACGAGA CCGATCCTCA TCAGCTCCCA
ATGTGCATAT AAACACAATA GAACCTGTCA ATATTGATGA
CTTGATTAGA GACCAAGGAT TTCGTGGTGA TGGAGGATCA
ACCACAGGTT TGTCTGCTAC CCCCCCTGCC TCATTACCTG
GCTCACTAAC TAACGTGAAA GCCTTACAGA AATCTCCAGG
ACCTCAGCGA GAAAGGAAGT CATCTTCATC CTCAGAAGAC
AGGAATCGAA TGAAAACACT TGGTAGACGG GACTCGAGTG
ATGATTGGGA GATTCCTGAT GGGCAGATTA CAGTGGGACA
AAGAATTGGA TCTGGATCAT TTGGAACAGT CTACAAGGGA
AAGTGGCATG GTGATGTGGC AGTGAAAATG TTGAATGTGA
CAGCACCTAC ACCTCAGCAG TTACAAGCCT TCAAAAATGA
AGTAGGAGTA CTCAGGAAAA CACGACATGT GAATATCCTA
CTCTTCATGG GCTATTCCAC AAAGCCACAA CTGGCTATTG
TTACCCAGTG GTGTGAGGGC TCCAGCTTGT ATCACCATCT
CCATATCATT GAGACCAAAT TTGAGATGAT CAAACTTATA
GATATTGCAC GACAGACTGC ACAGGGCATG GATTACTTAC
ACGCCAAGTC AATCATCCAC AGAGACCTCA AGAGTAATAA
TATATTTCTT CATGAAGACC TCACAGTAAA AATAGGTGAT
TTTGGTCTAG CTACAGTGAA ATCTCGATGG AGTGGGTCCC
ATCAGTTTGA ACAGTTGTCT GGATCCATTT TGTGGATGGC
ACCAGAAGTC ATCAGAATGC AAGATAAAAA TCCATACAGC
TTTCAGTCAG ATGTATATGC ATTTGGGATT GTTCTGTATG
AATTGATGAC TGGACAGTTA CCTTATTCAA ACATCAACAA
CAGGGACCAG ATAATTTTTA TGGTGGGACG AGGATACCTG
TCTCCAGATC TCAGTAAGGT ACGGAGTAAC TGTCCAAAAG
CCATGAAGAG ATTAATGGCA GAGTGCCTCA AAAAGAAAAG
AGATGAGAGA CCACTCTTTC CCCAAATTCT CGCCTCTATT
GAGCTGCTGG CCCGCTCATT GCCAAAAATT CACCGCAGTG
CATCAGAACC CTCCTTGAAT CGGGCTGGTT TCCAAACAGA
GGATTTTAGT CTATATGCTT GTGCTTCTCC AAAAACACCC
ATCCAGGCAG GGGGATATGG TGCGTTTCCT GTCCACTGAA
ACAAATGAGT GAGAGAGTTC AGGAGAGTAG CAACAAAAGG
AAAATAAATG AACATATGTT TGCTTATATG TTAAATTGAA
TAAAATACTC TCTTTTTTTT TAAGGTGGAA AAAAAAAAAA
AAAAAAAAAA AAAAAAAAAA AAAAAAACCC

A nucleic acid molecule can encode the full length B-Raf polypeptide or alternatively can encode a peptide fragment of B-Raf that is sufficient to confer the ability to proliferate on an anergized T cell, or that activates extracellular signal-regulated kinase (ERK). The nucleic acid can encode the naturally occurring B-Raf or fragment thereof, a conservative variant thereof (see below), or a modified form of the B-raf polypeptide or fragment thereof, as long as expression of the fragment or variant retains a function of B-Raf, such that it allows an anergized T cell to proliferate or activates ERK.

In one embodiment, the method includes the use of all fragments, mutants, or variants (e.g., modified forms) of B-Raf protein that retains a function of B-Raf, such as the ability to induce an anergized T cell to proliferate or the activation of ERK.

Thus, a protein is included that shares a significant homology with the natural B-Raf polypeptide and is capable of inducing proliferation in an anergized T cell. One skilled in the art can select such forms of B-Raf polypeptide based on their ability to induce T cells to proliferate upon introduction of a nucleic acid encoding the B-Raf protein in the T cell. The ability of a specific form of B-Raf to induce proliferation can be determined, for example by transfecting a nucleic acid encoding the specific form of B-Raf into a T cell, such that the B-Raf protein is synthesized in the T cells, and evaluating the proliferation rate of the T cells.

Furthermore, changes in the primary amino acid sequence of B-Raf are likely to be tolerated without significantly impairing the ability of the B-Raf molecule to induce proliferation of a T cell or to activate ERK. Accordingly, mutant forms of B-Raf that have amino acid substitutions, deletions and/or additions as compared to the naturally occurring amino acid sequence of a B-Raf molecule yet still retain the functional activity of the natural form of B-Raf are included. In one embodiment, conservative amino acid substitutions are made at one or more amino acid residues in a B-Raf polypeptide.

A conservative amino acid substitution is a substitution that does not change a function of B-Raf. In one embodiment, a conservative substitution is a substitution in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine).

To express a nucleic acid molecule encoding B-Raf in a T cell the nucleic acid can be operably linked to regulatory elements. Regulatory sequences are selected to direct expression of the desired protein in an appropriate T cell. Thus, regulatory sequences include promoters, enhancers and other expression control elements. Such regulatory sequences are known to those skilled in the art and are further described in Goeddel, Gene Expression Technology: Methods in Enzymology 185, Academic Press, San Diego, Calif., 1990. These regulatory elements include those required for transcription and translation of the nucleic acid encoding B-Raf, and include promoters, enhancers, polyadenylation signals, and sequences necessary for transport of the molecule to the appropriate cellular compartment. When the nucleic acid is a cDNA, and the vector is a recombinant expression vector, the regulatory functions responsible for transcription and/or translation of the cDNA are often provided by viral sequences. Examples of commonly used viral promoters include those derived from polyoma, Adenovirus 2, cytomegalovirus and Simian Virus 40, and retroviral LTRs.

Regulatory sequences operably linked to the cDNA encoding B-Raf can be selected to provide constitutive or inducible transcription. Inducible transcription can be accomplished by, for example, use of an inducible enhancer. Thus, in a specific embodiment, the nucleic acid molecule encoding B-Raf is operably linked to an inducible control element. In this manner, expression of B-Raf is regulated by an agent which affects the inducible control element (e.g., expression can be modulated by modulating the concentration of the inducing agent in the presence of the T cell). Inducible regulatory systems for use in mammalian cells are known in the art, for example systems in which gene expression is regulated by heavy metal ions (Mayo et al., Cell 29:99-108, 1982; Brinster et al., Nature 296:39-42, 1982; Searle et al., Mol. Cell. Biol. 5: 1480-1489, 1985), heat shock (Nouer et al., in Heat Shock Response, e.d. Nouer, L., CRC, Boca Raton, Fla., pp167-220, 1991), hormones (Lee et al., Nature 294:228-232, 1981; Hynes et al., Proc. Natl. Acad. Sci. USA 78:2038-2042, 1981; Klock et al., Nature 329:734-736, 1987) or tetracycline (Gossen, M. and Bujard, H., Proc. Natl. Acad Sci. USA 89:5547-5551, 1992). Other systems providing inducible gene expression controlled by contacting the T cells with specific inducing agents are described in published PCT Application No. WO 94/18317 and published PCT Application No. WO 93/23431.

Inducible control elements may function in all T cells, or alternatively, only in a specific subset of T cells, such as in CD4+ T cells, CD8+ T cells, T helper 1 (Th1), T helper 2 (Th2) cells. Inducible control elements can also be selected which are regulated by one agent in one type of T cells (such as CD4+ T cells) yet which are regulated by another agent in another type of T cells (such as CD8+ T cells).

In another embodiment, a nucleic acid molecule that encodes a B-Raf polypeptide is under the control of a constitutive regulatory sequence. In one embodiment, a constitutive regulatory element is a viral promoter. Examples of commonly used viral promoters include those, derived from polyoma, Adenovirus 2, cytomegalovirus, Simian Virus 40, and retroviral LTRs. Alternatively, T cell-specific enhancers can be used, e.g. T cell receptor enhancers (see e.g. Winoto and Baltimore, EMBO J. 8:729-733, 1989). Constitutive control elements may function in all T cells, or alternatively, only in a specific subset of T cells, such as in CD4+ T cells, CD8+ T cells, T helper 1 (Th1), T helper 2 (Th2) cells. Exemplary, non-limiting control elements for expression in CD4+ T cells are disclosed in the examples section below.

In one embodiment, a nucleic acid molecule encoding a B-Raf polypeptide, operably linked to a regulatory element, is included in a vector. Examples of vectors include plasmids, viruses or other nucleic acid molecules comprising, for example, sequences that are necessary for selection and amplification of the nucleic acid molecule in bacteria.

The nucleic acid molecule encoding a B-Raf polypeptide can be introduced into a T cell by various methods typically referred to as transfection. Suitable transfection methods include, but are not limited to, electroporation, calcium-phosphate precipitation, DEAE-dextran treatment, lipofection, microinjection, and viral infection. Suitable methods for transfecting mammalian cells can be found in Sambrook et al. (Molecular Cloning: A Laboratory Manual. 2nd Edition. Cold Spring Harbor Laboratory press, 1989) and other laboratory textbooks.

In one embodiment, the nucleic acid molecule encoding a B-Raf polypeptide is introduced into a T cell using a viral vector. Such viral vectors include, but are not limited to, recombinant retroviruses, adenovirus, adeno-associated virus, and herpes simplex virus-1.

In one specific, non-limiting example, a retrovirus vector or an adeno-associated virus vectors is utilized to introduce a nucleic acid encoding B-Raf into a T cell either in vivo or in vitro. These vectors provide efficient delivery of genes into T cells, and the transferred nucleic acids are stably integrated into the chromosomal DNA of the host cell. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A.D., Blood 76:271, 1990). Thus, recombinant retrovirus can be constructed in which part of the retroviral coding sequence (gag, pol, env) has been replaced by nucleic acid encoding a B-Raf polypeptide rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, Sections 9.10-9.14, 1989, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include .psi.Crip, .psi.Cre, .psi.2 and .psi.Am.

Furthermore, it has been shown that it is possible to limit the infection spectrum of retroviruses, and consequently of retroviral-based vectors, by modifying the viral packaging proteins on the surface of the viral particle (see, for example PCT publications WO 93/25234 and WO 94/06920). For instance, strategies for the modification of the infection spectrum of retroviral vectors include: coupling antibodies specific for cell surface antigens to the viral env protein (Roux et al., PNAS 86:9079-9083, 1989; Julan et al., J. Gen Virol 73:3251-3255, 1992; and Goud et al., Virology 163:251-254, 1983); or coupling cell surface receptor ligands to the viral env proteins (Neda et al., J. Biol Chem 266:14143-14146, 1991). Coupling can be in the form of the chemical cross-linking with a protein or other variety (e.g. lactose to convert the env protein to an asialoglycoprotein), as well as by generating fusion proteins (e.g. single-chain antibody/env fusion proteins). Thus, in one embodiment, viral particles containing a nucleic acid molecule encoding a B-Raf polypeptide are modified, for example, according to the methods described above, such that they can specifically target subsets of T cells. For example, the viral particle can be coated with antibodies to surface molecule that are specific to certain types of T cells. In particular, it is possible to selectively target CD4+ T cells by linking to the viral particle antibodies that recognize the CD4 molecule on the T cell. Thus, infection of CD4+ T cells will occur preferentially over infection of CD8+ T cells. This method is particularly useful when proliferation of only specific subsets of T cells is desired. Moreover, it may be desirable to limit the introduction of the nucleic acid molecule encoding a B-Raf polypeptide to T cells or specific subsets for use in vivo.

Additional retroviral systems for introducing and expressing a nucleic acid molecule encoding a B-Raf polypeptide in T cells including primary T cells are described in Kasid et al., Proc. Natl. Acad. Sci. USA 87:473, 1990; Morecki et al., Cancer Immunol. Immunother. 32:342, 1991; Culver et al., Proc. Natl. Acad. Sci USA 88:3155, 1991; and Finer et al., Blood 83:43, 1994.

Another viral gene delivery system of use is adenovirus-derived vectors. The genome of an adenovirus can be manipulated such that it encodes and expresses a gene product of interest but is inactivated in terms of its ability to replicate in a normal lytic viral life cycle (Berkner et al., Bio Techniques 6:616, 1988; Rosenfeld et al., Science 252:431-434, 1991; Rosenfeld et al., Cell 68:143-155, 1992). In one specific, non-limiting example, suitable adenoviral vectors derived from the adenovirus strain Ad type 5 dl324 or other strains of adenovirus (e.g., Ad2, Ad3, Ad7 etc.) are known in the art. Recombinant adenoviruses can be advantageous in certain circumstances in that they are not capable of infecting non-dividing cells. Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Haj-Ahmand and Graham, J. Virol. 57:267, 1986). In one embodiment, the adenoviral vector is deleted for all or parts of the viral E1 and E3 genes but retain as much as 80% of the adenoviral genetic material (see, e.g., Jones et al., Cell 16:683, 1979; Graham et al. in Methods in Molecular Biology, E. J. Murray (ed.) Humana, Clifton, N.J., vol. 7. pp. 109-127, 1991). In another embodiment, the E3, E3, and/or E4 genes are also deleted (a “gutless” adenoviral vector). Expression of the inserted nucleic acid molecule encoding a B-Raf polypeptide can be under control of, for example, the E1A promoter, the major late promoter (MLP) and associated leader sequences, the E3 promoter, or exogenously added promoter sequences.

Yet another viral vector system useful for in vivo delivery of a nucleic acid molecule encoding a B-Raf polypeptide is the adeno-associated virus (AAV). Adeno-associated virus is a naturally occurring defective virus that requires another virus, such as an adenovirus or a herpes virus, as a helper virus for efficient replication and a productive life cycle(for review see Muzyczka et al., Curr. Topics in Micro. and Immunol. 158:97-129, 1992). Adeno-associated virus is also one of the few viruses that can integrate its DNA into non-dividing cells, and exhibits a high frequency of stable integration (see for example Samulski et al., J. Virol. 63:3822-3828, 1989; McLaughlin et al., J. Virol. 62:1963-1973, 1989). Vectors containing as few as 300 base pairs of AAV can be packaged and can integrate. A variety of nucleic acids have been introduced into different cell types using AAV vectors (see, for example, Hermonat et al., Proc. Natl. Acad Sci. USA 81:6466-6470, 1984; Tratschin et al., Mol. Cell. Biol. 4:2072-2081, 1985; Tratschin et al., J. Virol. 51:611-619, 1984). Other viral vector systems are of use include, but are not limited to vaccinia virus vectors.

In addition to encoding a B-Raf polypeptide, an expression vector can also encode a selectable marker. In one embodiment, the selectable marker is a protein that confers resistance to drugs such as G418, hygromycin and methotrexate. Selectable markers can be introduced on the same vector (e.g. plasmid) as the nucleic acid molecule encoding a B-Raf polypeptide or may be introduced on a separate vector (e.g. plasmid).

Another targeted delivery system for polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. One colloidal system is a liposome. Liposomes are artificial membrane vesicles that are useful as delivery vehicles in vitro and in vivo. It has been shown that large uni-lamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley et al., 1981, Trends Biochem. Sci. 6:77, 1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino et al., Biotechniques 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidyl-glycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

The nucleic acid molecule encoding a B-Raf polypeptide can be delivered into a T cell using other cell-delivery vehicle such as derivatized (e.g. antibody conjugated) polylysine conjugates, gramicidin S, and artificial viral envelopes. These vehicles can deliver a nucleic acid encoding a B-Raf polypeptide that is included in a vector e.g. a plasmid or virus DNA. In a specific embodiment, efficient gene expression in primary T lymphocytes, in particular in CD3+, CD4+ and/or CD8+ T cells, is obtained using adeno-associated virus plasmid DNA complexed to cationic liposomes (see Philip et al., Mol. Cell. Biol. 14:2411, 1994).

In another embodiment, a nucleic acid molecule encoding a B-Raf polypeptide is delivered into a specific cell in the form of a soluble molecular complex. The complex contains the nucleic acid release-ably bound to a carrier made of a nucleic acid binding agent and a cell-specific binding agent which binds to a surface molecule of the specific cell and is of a size that can be subsequently internalized by the cell. Such complexes are described in U.S. Pat. No. 5,166,320.

In another embodiment, the nucleic acid encoding a B-Raf polypeptide is introduced into T cells by particle bombardment (see Yang and Sun, Nature Medicine 1:481, 1995; U.S. Pat. No. 6,143,291).

In a further embodiment, the nucleic acid encoding B-Raf is delivered to a T cell in conjunction with a co-stimulatory agent, such as an agent that cross-links CD28. “In conjunction” refers to both concurrent and sequential administration of an agent and the nucleic acid encoding B-Raf. An agent that cross-links CD28 can be a ligand of CD28 or CTLA4, such as a B-lymphocyte antigen B7-1 or B7-2 molecules, fragments thereof, or modifications thereof, which are capable of providing costimulatory signals to the T cells. Appropriate forms of natural ligands of CD28 can be identified by, for example, contacting activated T cells with a form of a natural ligand of CD28 and performing a standard T cell proliferation assay. Stimulatory forms of natural ligands of CD28/CTLA4 are described, for example, in PCT Publication No. WO 95/03408. In another embodiment, the agent that cross-links CD28 is a monoclonal antibody that specifically binds CD28. In another embodiment, the natural ligand for CD28 is used, such as B7. In specific, non-limiting examples the ligand for CD28 is B7.1 or B7.2 ( see Sharpe and Freeman, Nat Rev Immunol 2(2):116-26, 2002).

In another embodiment, the nucleic acid encoding B-Raf is delivered to a T cell in conjunction with an agent that signals the T cell receptor (TCR)/CD3 complex. In a specific non-limiting example, the agent which stimulates the T cell receptor or the CD3 complex associated with the T cell receptor. is an antibody that specifically binds CD3, such as the monoclonal antibody OKT3 (available from the American Type Culture Collection, Rockville, Md.; No. CRL 8001). In yet another embodiment, the nucleic acid encoding B-Raf is delivered to a T cell in conjunction with an activating signal provided by an antigen or an antigen presenting cell. Thus, it is possible to selectively stimulate proliferation in a population of T cells by contacting the T cells with one or more antigens or one or more antigen presenting cells.

### Inhibition of Rap-1 Activity By Other Agents

A method is provided herein for inducing an anergized T cell to proliferate, wherein the method includes providing the anergized T cell with an effective amount of a composition that inhibits the expression or activity of Rap-1. Methods for reducing Rap-1 protein levels in a T cell include methods comprising contacting a T cell with an agent which decreases the expression of Rap-1. An agent that decreases expression of Rap-1 including an agent which destabilizes mRNA encoding Rap-1, an agent which blocks splicing of the mRNA encoding Rap-1, and an agent which blocks translation of the mRNA, or any combination of these agents.

In one embodiment, antisense nucleic acids that inhibit production of Rap-1 protein are introduced into the T cells. An “antisense” nucleic acid comprises a nucleotide sequence which is complementary to a “sense” nucleic acid, e.g., complementary to an mRNA sequence encoding a protein, constructed according to the rules of Watson and Crick base pairing. Accordingly, an antisense nucleic acid can hydrogen bond to a sense nucleic acid. An antisense sequence complementary to a sequence of an mRNA can be complementary to a sequence found in the coding region of the mRNA or can be complementary to a 5′ or 3′ untranslated region of the mRNA. In one specific, non-limiting example, an antisense nucleic acid is complementary to a region preceding or spanning the initiation codon or in the 3′ untranslated region of an mRNA. An antisense nucleic acid can be designed based upon the nucleotide sequence shown in published PCT Application No. 9611562, herein incorporated by reference, or another Rap-1-encoding sequence known in the art. For example, a nucleic acid is designed which has a sequence complementary to the coding or untranslated region of the nucleotide sequence of GenBank Accession No. A80086, or SEQ ID NO:1 of published PCT Application No. WO9611562, both of which are herein incorporated by reference. In one embodiment, an antisense nucleic acid that specifically binds Rap-1 has the following sequence:

(SEQ ID NO:3)

CCCTGAGGCA AGTCTGGGTA CGTAACGTAT AAAGCAACAG
CAAATGAAAT CTGAATGCGG GAATGACAAC TGGACTTCGA
AACATAATTT AATATTCATG AAATTGCACA CATACCAATA
GTATTCTATG TCTCGTCTAG CCTCTTAATC CAACTATCTC
AACTGCCTTG CAGTTTGCCT ACCAGACACC CCAACTCTCT
ACATACATTC TGTCATGTAA ATGCTGACCT TCTGACTGAA
ATTTAATAAA TTAACATGGG ATTTACATCA AGGGATTTTT
GTCTGGTGAG TGCATTGCCA GAAAGCATGC CTAGTTCCTG AC

Antisense nucleic acids can be constructed using chemical synthesis and enzymatic ligation reactions using procedures known in the art. The antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Antisense oligonucleotides can be introduced into a T cell in culture to inhibit expression of Rap-1. One or more antisense nucleic acids, such as oligonucleotides, can be added to cells in culture media, typically at about 200 μg/ml.

Alternatively, the antisense nucleic acid can be produced biologically in the T cell using an expression vector into which a nucleic acid corresponding to at least a fragment of a nucleotide sequence encoding a Rap-1 protein has been subcloned in an antisense orientation (i.e., RNA transcribed from the inserted nucleic acid will be of an antisense orientation to a target nucleic acid of interest). The antisense expression vector can be in the form of a recombinant plasmid, phagemid or attenuated virus in which antisense nucleic acids are produced under the control of a high efficiency regulatory region. The regulatory region can stimulate constitutive or inducible expression of the nucleic acid molecule. The regulatory regions controlling the expression of the nucleic acid molecule, and the vectors that carry such sequences, as well as methods for introducing the nucleic acid molecule into the T cells have been described above. A nucleic acid molecule encoding Rap-1 antisense mRNA can be introduced into T cells in vitro or in vivo. Methods for introducing nucleic acids into T cells in vivo are also described above (for a discussion of the regulation of gene expression using antisense genes see Weintraub et al., Reviews—Trends in Genetics, Vol. 1(1) 1986)

In another embodiment, Rap-1 protein levels in a T cell are reduced by introducing into the T cell a nucleic acid encoding a form of antisense nucleic acid which is a ribozyme. Ribozymes are catalytic RNA molecules with ribonuclease activity which are capable of cleaving a single-stranded nucleic acid, such as an mRNA, to which they have a complementary region. A ribozyme having specificity for a Rap-1-encoding sequence can be designed based upon the nucleotide sequence of a Rap-1-encoding mRNA. For example, a derivative of a Tetrahymena L-19 IVS RNA can be constructed in which the base sequence of the active site is complementary to the base sequence to be cleaved in a Rap-1 encoding mRNA (see U.S. Pat. No. 4,987,071 and U.S. Pat. No. 5,116,742). Alternatively, a Rap-1 encoding sequence can be used to select a catalytic RNA having a specific ribonuclease activity from a pool of RNA molecules (see for example Bartel et al., Science 261:1411-1418, 1993).

### Methods of Treatment

In one embodiment, a method is provided for inducing a response against an antigen in a subject. The method includes administering a therapeutically effective amount of a nucleic acid sequence comprising a promoter operably linked to a nucleic acid sequence encoding B-Raf, thereby inducing the immune response against the antigen. By subject is meant any mammal, including a human. In one embodiment, the subject has a neoplasm, such as a breast, prostate, liver, lung, ovarian, testicular, colon, or skin tumor. In one specific, non-limiting example, the antigen is a tumor antigen, such as prostate specific antigen or carcinoembryonic antigen.

In another embodiment, the method includes administering a pharmaceutical composition including a nucleic acid encoding B-Raf or a functional fragment or variant thereof and a pharmaceutically acceptable carrier. The delivery of the pharmaceutical composition can be accomplished by any means known to the skilled artisan. In a further embodiment, a method is provided that includes administering a therapeutically effective amount of a composition that inhibits the expression or activity of Rap-1, thereby inducing the immune response against the antigen. The agent that inhibits Rap-1 can be administered as a pharmaceutical composition.

The pharmaceutical compositions are prepared and administered in dose units. Solid dose units are tablets, capsules and suppositories. For treatment of a subject, depending on activity of the compound, manner of administration, nature and severity of the disorder, age and body weight of the patient, different daily doses are necessary. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions are in general administered by subcutaneous or intramuscular injection, or as implants, but any mode of administration is possible in principle, as long as the nucleic acids are taken up by dendritic cells. Suitable solid or liquid pharmaceutical preparation forms are, for example, granules, powders, tablets, coated tablets, (micro)capsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science 249:1527-1533, 1990, which is incorporated herein by reference.

The pharmaceutical compositions may be administered locally or systemically. Amounts effective for therapeutic use will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, e.g., in Gilman et al., eds., Goodman and Gilman. The Pharmacological Bases of Therapeutics, 8th ed., Pergamon Press, 1990; and Remington's Pharmaceutical Sciences, 17th ed., Mack Publishing Co., Easton, Pa., 1990, each of which is herein incorporated by reference.

Effective doses of the therapeutic molecules will vary depending on the nature and severity of the condition to be treated, the age and condition of the patient and other clinical factors. Thus, the final determination of the appropriate treatment regimen will be made by the attending clinician. The dosing schedule may vary from once a week to daily depending on a number of clinical factors. In the case of a more aggressive disease it may be desirable to administer doses such as those described above by subcutaneous administration. Continuous infusion may also be appropriate.

For administration to animals, purified therapeutically active molecules, such as nucleic acids encoding a polypeptide of interest, are generally combined with a pharmaceutically acceptable carrier. Pharmaceutical preparations may contain only one type of therapeutic molecule, or may be composed of a combination of several types of therapeutic molecules. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pill-based forms of pharmaceutical proteins may also be administered subcutaneously, particularly if formulated in a slow-release composition. Slow-release formulations may be produced by combining the target protein with a biocompatible matrix, such as cholesterol. Another possible method of administering pharmaceuticals is through the use of mini osmotic pumps. As stated above a biocompatible carrier would also be used in conjunction with this method of delivery.

The pharmaceutical compositions may be administered by any means that achieve their intended purpose. Amounts and regimens for the administration of the therapeutic molecules can be determined readily by those with ordinary skill in the clinical art of treating diseases. Typical amounts initially administered would be those amounts adequate to achieve tissue concentrations at the site of action which have been found to achieve the desired effect in an animal model.

### Screening Methods

A method is provided herein for screening for an agent that activates B-raf. The method includes contacting an anergized T cell with the agent and detecting proliferation of the T cell, thereby determining if the agent activates B-raf. The proliferation of the T cell can then be compared to the proliferation of a control not incubated with the agent. Alternatively, the effect of the compound on a signaling event (e.g., ERK activity) can be evaluated.

The agents which affect B-Raf include peptides, polypeptides, pepidomimentics, chemical compounds, nucleic acids, and biological agents. “Incubating” includes conditions which allow contact between the test agent and the T cell or a component of the T cell. “Contacting” includes in solution and solid phase. The test agent may also be a combinatorial library for screening a plurality of compounds. Agents identified in the methods disclosed herein can be further evaluated, detected, cloned, sequenced, and the like, either in solution of after binding to a solid support, by any method usually applied to the detection of a specific DNA sequence, such as PCR, oligomer restriction (Saiki et al., Bio/Technology 3:1008-1012, 1985), allele-specific oligonucleotide (ASO) probe analysis (Conner et al., Proc. Natl. Acad. Sci. USA 80:278, 1983), oligonucleotide ligation assays (OLAs) (Landegren et al., Science 241:1077, 1988), and the like. Molecular techniques for DNA analysis have been reviewed (Landegren et al., Science 242:229-237, 1988).

If the agent binds B-Raf directly, the binding affinity can also be determined in either cells or a cell-free preparation including B-Raf. In these assays, a labeled ligand, such as labeled B-Raf, is employed. A number of labels have been indicated previously (e.g., radiolabels, fluorescence labels, among others) to be of use. The candidate compound is added in an appropriate buffered medium. After an incubation to ensure that binding has occurred, any nonspecifically bound components of the assay medium can be washed off, and the amount of label bound to the agent can be determined. The label may be quantitatively measured. By using standards, the relative binding affinity of a candidate compound can thus be determined.

### Transgenic Non-Human Animals

Another embodiment relates to transgenic animals having cells that express B-Raf. Such transgenic animals represent a model system for the study of anergy, and for and the induction of the proliferation of anergic T cells. These transgenic animals can be used to study agents that affect the proliferation of anergic T cells or the activation of extracellular signal-regulated kinases.

A transgenic non-human mammal denotes all mammalian species except human. An individual animal in all stages of development, including embryonic and fetal stages are included. Farm animals (pigs, goats, sheep, cows, horses, rabbits and the like), rodents (such as mice), and domestic pets (for example, cats and dogs) are also included. In one embodiment, the transgenic non-human animal is a mouse.

A “transgenic” animal is any animal containing cells that bear genetic information received, directly or indirectly, by deliberate genetic manipulation at the subcellular level, such as by microinjection or infection with recombinant virus. “Transgenic” in the present context does not encompass classical crossbreeding or in vitro fertilization, but rather denotes animals in which one or more cells receive a recombinant DNA molecule. Although this molecule can be integrated within the animal's chromosomes, the use of extrachromosomally replicating DNA sequences, such as might be engineered into yeast artificial chromosomes, is also contemplated.

The term “transgenic animal” also includes a “germ cell line” transgenic animal. A germ cell line transgenic animal is a transgenic animal in which the genetic information has been taken up and incorporated into a germ line cell, therefore conferring the ability to transfer the information to offspring. If such offspring in fact possess some or all of that information, then they, too, are transgenic animals. In one embodiment, the transgenic are produced by introducing into single cell embryos DNA encoding B-Raf in a manner such that the polynucleotides are stably integrated into the DNA of germ line cells of the mature animal and inherited in normal Mendelian fashion. Advances in technologies for embryo micromanipulation now permit introduction of heterologous DNA into fertilized mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In one embodiment, developing embryos are infected with a retrovirus containing the desired DNA, and transgenic animals produced from the infected embryo.

In another embodiment the appropriate DNAs are coinjected into the pronucleus or cytoplasm of embryos, preferably at the single cell stage, and the embryos allowed to develop into mature transgenic animals. These techniques are well known. For instance, reviews of standard laboratory procedures for microinjection of heterologous DNAs into mammalian (mouse, pig, rabbit, sheep, goat, cow) fertilized ova include: Hogan et al., Manipulating the Mouse Embryo, Cold Spring Harbor Press, 1986; Krimpenfort et al., Bio/Technology 9:86, 1991; Palmiter et al. Cell 41:343, 1985; Kraemer et al., Genetic Manipulation of the Early Mammalian Embryo, Cold Spring Harbor Laboratory Press, 1985; Hammer et al., Nature 315:680, 1985; Purcel et al., Science 244:1281, 1986; U.S. Pat. No. 5,175,385; U.S. Pat. No. 5,175,384.

The cDNA that encodes a B-Raf polypeptide can be fused in proper reading frame under the transcriptional and translational control of a vector to produce a genetic construct that is then amplified, for example, by preparation in a bacterial vector, according to conventional methods. See, for example, Sambrook et al., Molecular Cloning: a Laboratory Manual, Cold Spring Harbor Press, 1989. In one embodiment, a nucleic acid encoding B-Raf, operably linked to a promoter, such as a CD4 promoter, is included in the vector. The amplified construct is thereafter excised from the vector and purified for use in producing transgenic animals.

The disclosure is illustrated by the following non-limiting Examples.

### EXAMPLES

### Example 1

### Materials and Methods

Jurkat Cell Culture, Transfection and Stimulation. Cells from the human T cell leukemia cell line Jurkat (clone E6-1, from A.T.C.C., Manassas, Va.) were maintained in RPMI-1640 media (Gibco, Bethesda, Md.) containing 10% FCS (Hyclone, Logan, N.J.) at 37° C. with 5% CO2. For transient transfections, 5×10⁷ Jurkat T cells were transfected by electroporation (250V/950 μF) with 10 μg RapV12 (constitutively active Rap1) and 10 μg Raf-1 or 10 μg B-Raf plasmids. All cells were transfected with the reporter constructs, Gal4-Elk1 and 5×Gal4-E1b/luciferase (3 μg each) and activity was measured by luciferase assay as previously described (Vossler et al., Cell 89:73-82, 1997). The total DNA transfected was held constant with the addition of pcDNA3 (vector) and after transfection, cells recovered for 24 h in media. Prior to lysis, 10⁶ cells were stimulated with anti-TCR antibody (clone C305, 1/40 hybridoma supernatant; gift from A. Weiss, UCSD, CA).

Transgenic Mice. The cDNA encoding B-Raf was placed downstream of the CD4 promoter under control of a T cell specific enhancer element with the cis-acting silencer element deleted (Shinichiro et al., Mol. Cell. Biol. 11:5506-5515, 1991). The full-length cDNAs encoding B-Raf and myc-RapV12 were inserted into a unique XhoI site downstream of this promoter. Over 100 embryos were injected with each construct and implanted into pseudo-pregnant mothers. Litters were genotyped using both tail and ear DNA by Southern blot using random-primed probes derived from the full-length cDNAs and by PCR analysis using vector specific primers. The founders that were identified by these methods were mated with wild type C57BL/6 mice and F1 litters genotyped. Experiments were performed on adult mice 8-10 weeks of age. For anergy experiments B-Raf transgenic animals were crossed with the TCR transgenic mouse line, AD10 (Kaye et al., J. Immunol. 148:3342-3353, 1992). A large percentage (80-90%) of the T cells from the AD10 TCR transgenic mice expressed a recombined TCR, which recognizes pigeon cytochrome c (PCC) peptide (amino acids 88-104) presented by MHC class II I-E^{k}. B-Raf transgenic mice were also crossed with the TCR transgenic mouse line, AND. The T cells from the AND mouse recognize the PCC peptide presented byI-A^{b} (Kaye et al., J. Immunol. 148:3342-3353, 1992). In all animal studies, experiments were performed in compliance with the relevant laws and institutional guidelines and were approved by the IACUC.

Immunohistochemistry. Thymus, spleen and lymph nodes were taken from B-Raf transgenic and wild type animals. The tissues were fixed immediately in 4% paraformaldehyde on ice for 4 hours. Following fixation, tissues were immersed in a 30% sucrose solution at 4° C. for 12 hours. Tissues were then embedded in OCT solution (Tissue-Tek, Torrance, Calif.) and 20 μM sections were cut. Sections were stained with anti-B-Raf antibody(1/500 Santa Cruz Biotechnology Inc., Santa Cruz, Calif.) followed by HRP-conjugated anti-goat antibody (1/500) and staining visualized by DAB color reaction.

Flow Cytometry. The surface expression of CD8 and CD4, molecules was analyzed by direct staining with FITC labeled anti-CD8 mAb (53-6.7, PharMingen) and Cy-Chrome labeled anti-CD4 mAb (RM4-5, Pharmingen). CD69 expression on TCR transgenic T cells was analyzed by staining by staining with FITC labeled anti-Vα11 Ab (RR8-1, PharMingen), PE labeled anti-Vβ3 Ab (KJ25, Pharmingen) and biotinylated anti-CD69 mAb (H1.2F3, PharMingen) followed by streptavidin APC (PharMingen). Intracellular staining for B-Raf expression was performed using the cytofix/cytoperm kit (Pharmingen) according to the manufacturers instructions. Following surface staining for CD4 and CD8, cells were fixed and permeabilized for 30 minutes at room temperature. After washing twice cells were incubated with 20 μg/ml anti-B-Raf antibody (c-19, Santa Cruz Biotechnology Inc., Santa Cruz, Calif.) followed by Cy5-labeled anti-rabbit Ab (Jackson Immuno Reseaarch Laboratories Inc., West Grove, Pa.).

The stained cells were analyzed by flow cytometry using a FACSCalibur (Becton Dickinson, Mountain View, Calif.). Viable cells were gated using forward and side scatter. The gated cells were analyzed for expression of CD8, CD4 or CD69.

Primary T Cell Isolation and Stimulation. T cells from the spleen or lymph nodes were purified on a murine T cell enrichment column (R&D Systems, Minneapolis, Minn.) according to the manufacturer's instructions. Ten million T cells were incubated with 0.5 μg/ml anti-CD3 antibody (clone 145-2C11, Pharmingen, San Diego, Calif.) and/or 10 μg/ml anti-CD28 antibody (clone 37.51, Pharmingen, San Diego, Calif.) at 4° C. for 30 minutes, washed, then stimulated with the addition of 10 μg/ml of goat anti-hamster immunoglobulin (Southern Biotechnology Assoc. Inc., Birmingham, Ala.) for 5 minutes at 37° C.

T Cell Anergy. The protocol for in vitro anergy was modified from Schwartz (Jenkins et al., Proc. Natl. Acad. Sci. USA 84:5409-5413 (1987). Splenocytes from AD10 transgenic and AD10×B-Raf transgenic mice were cultured 4-6 days in T cell media (RPMI-1640 (Gibco, Bethesda, Md.) containing 10% FCS (Hyclone, Logan, Utah) and supplemented with 1 mM L-glutamine, sodium pyruvate (100 mg/ml), 5×10⁻⁵ M β-mercaptoethanol, essential and non-essential amino acids (Gibco, Bethesda, Md.), 100U/ml Penicillin G, 100 U/ml Streptomycin and 50 μg/ml gentamycin antibiotics) in the presence of 2.5 μM PCC peptide. Live cells were recovered over a density gradient and 5×10⁶ cells/ml were incubated overnight with or without plate bound anti-TCRβ antibody (10 μg/ml, clone H57-957, Pharmingen). T cells were removed from the plates and rested for 24 hours prior to experimental use.

In vivo Rap1 and Ras Assays. This assay was performed as previously described (Carey et al., Mol. Cell. Biol. 20:8409-8419, 2000). Isolated T cells were stimulated as described above. Cells were lysed in ice cold Rap1 lysis buffer with inhibitors and the protein content of clarified lysates determined by BioRad assay. Three hundred μg of total cell lysate was incubated with 40 μg of glutathione s-transferase (Gst)-Ra1-GDS-Ras binding domain (RBD) fusion protein (gift of J. L. Bos, Utrecht University, Utrecht, The Netherlands) coupled to glutathione agarose beads for 1 hour at 4° C. to recover activated Rap1 (Rap-GTP). Beads were pelleted and washed 3 times lysis buffer, and the protein eluted from the beads by boiling in Laemelli buffer. Activated Ras (Ras-GTP) was isolated from cell lysates using agarose coupled Gst fused to the Ras-binding domain (RBD) of Raf-1 (Gst-Raf-RBD) provided in a Ras activation kit (Upstate Biotechnology, Inc., Lake Placid, N.Y.) according to the manufacturers instructions. Proteins were resolved by SDS-PAGE and detected by Western blot (see below).

Preparation of Nuclear Extracts. Control and anergic cells (2.5×10⁶/ml) were rested for 48 hours, then restimulated at 37° C. for 3 hours in 6 well plates coated with 2 μg/ml anti-CD3 and 10 μg/ml anti-CD28 mAbs (clones 145-2C11 and 37.51 respectively, PharMingen). Cells were then collected and washed with ice cold PBS and lysed in cytosolic buffer (10 mM HEPES (pH 7.8), 10 mM KCl, 2 mM MgCl2, 0.1 mM EDTA, 0.2% Nonidet P-40) containing 1 mM DTT, 0.5 mM PMSF, 1 μg/ml aprotinin and 5 μg/ml leupeptin for 5 minutes on ice. Nuclei were removed by centrifugation, washed once in cytosolic buffer, then lysed in hypertonic buffer (50 mM HEPES (pH 7.8), 50 mM KCl, 300 mM NaCl, 0.1 mM EDTA, 10% glycerol) containing 1 mM DTT, 0.5 mM PMSF, 1 μg/ml aprotinin and 5 μg/ml leupeptin for 30 minutes on ice and then centrifuged for 15 minutes at 14,000 rpm at 4° C. to remove the insoluble fraction. Proteins were resolved by SDS-PAGE and detected by Western blot (see below).

Western Blots. Proteins were separated in a 12% gel, followed by transfer to a polyvinylidine diflouride membrane. Membranes were blocked in 5% BSA and probed with either anti-Rap1 polyclonal antibody (anti-Krev-1, Santa Cruz Biotechnology Inc., Santa Cruz, Calif.) or anti-Ras mAb (clone 10, Upstate Biotechnology, Inc, Lake Placid, N.Y.) and then with a horseradish peroxidase-conjugated secondary antibody (Amersham, Piscata Way, N.J.). Proteins were detected by enhanced chemiluminesence. Phosphorylation of ERK1, ERK2, JNK1, and JNK2 were detected from 30 μg of total cell lysate by immunoblotting with phosopho-specific MAP kinase polyclonal antibodies (Cell Signaling Technology, Beverly, Mass.) and total ERKs and JNKs were detected by immunoblotting with anti-ERK and anti-JNK polyclonal antibodies (Santa Cruz Biotechnology Inc., Santa Cruz, Calif.). Fos levels were detected from 4 ug of total nuclear proteins by Western blot using anti-c-Fos Ab (H-125, Santa Cruz Biotechnology Inc., Santa Cruz, Calif.).

CD69 Expression. Anergic and non-anergic AD10 and AD 10×B-Raf T cells (10⁶ cells/ml) were stimulated with 2 μg/ml anti-CD3 and 10 μg/ml anti CD28 plate bound Abs (Pharmingen, San Diego, Calif. Pharmingen) for 18 hours at 37° C. Cells were harvested on ice and stained for CD69 expression with biotinylated anti-CD69 mAb (H1.2F3, PharMingen) followed by streptavidin APC (see Flow Cytometry).

T Cell Proliferation. Splenocytes at a concentration of 10⁶ cells/ml were plated into 96 well plates (100μl) in the presence of a range of concentrations of anti-CD3 antibody. Alternatively, purified anergic and control AD10 and AD10×B-Raf T cells (5×10⁵ cells/ml) were plated into 96 well plates with 2×10⁶ cells/ml irradiated B10BR splenocytes, as APCs in the presence of varying concentrations of PCC peptide. Assays were incubated for 24-72 hours at 37° C. and pulsed with 1 μCi of [³H] thymidine/well for the final 16-18 hours of culture. The plates were harvested using a Packard 96-well Filtermate Harvester and counted on a Packard Top Count Scintillation counter.

### Example 2

### Expression of B-Raf, But Not Raf-1, Overcomes Rap1's Ability To Block Elk-1 Activation

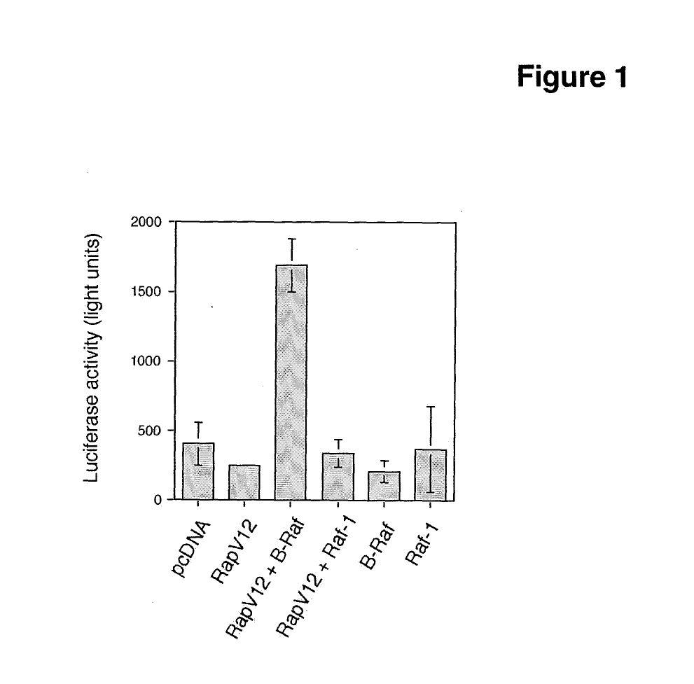

It has previously been shown that TCR stimulation of Jurkat T cells activates Rap1 to limit Ras-dependent signals to Raf-1 and ERKs (Carey et al., Mol. Cell. Biol 20:8409-8419 (2000). It has also been established that in B-Raf-expressing cells, Rap1 is a potent activator of ERKs (Schmitt et al., J. Biol. Chem. 275:25342-25350, 2000; Vossler et al., Cell 89:73-82, 1997). To test the ability of B-Raf to cooperate with RapV12, a constitutively active mutant of Rap1 to augment ERK signaling in T cells, ERK activity was measured in a transcription-coupled assay monitoring the ERK-induced phosphorylation of the transcription factor Elk-1 (Marais et al., Cell 73:381-393, 1993; Vossler et al., Cell 89:73-82, 1997). To examine whether B-Raf can convert RapV12 into an activator of Elk-1, Jurkat cells were transfected with RapV12 alone or with B-Raf or Raf-1. The data show that neither B-Raf nor RapV12 activated Elk-1 transcription when transfected alone. However, when transfected together, B-Raf and RapV12 co-operated to activate Elk-1. This action of B-Raf was specific for this isoform of the Raf family kinases, as co-transfection of Raf-1 with RapV12 did not enhance Elk-1 activity (FIG. 1).

### Example 3

### Transgenic Expression of RapV12 and B-Raf

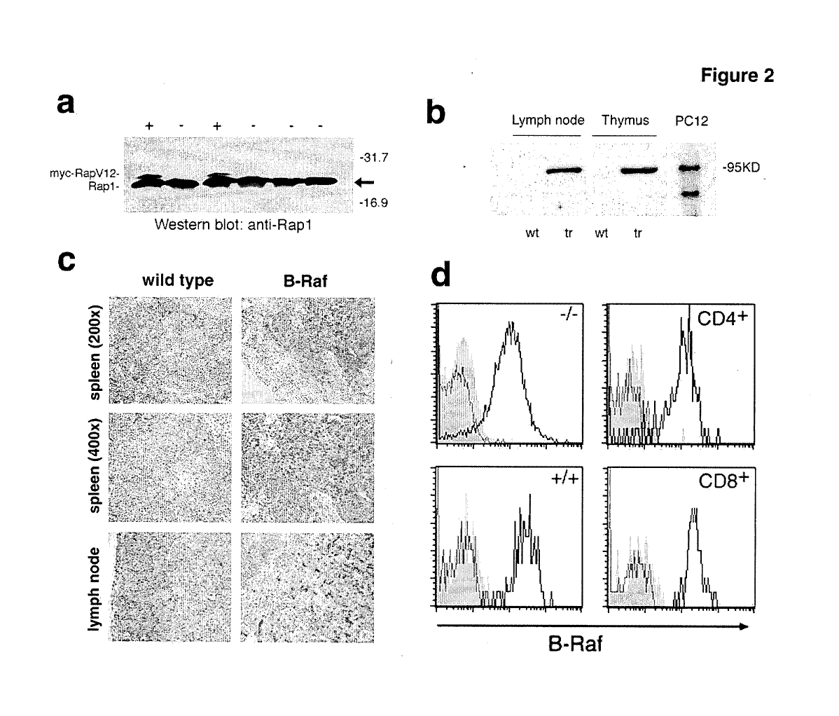

To further examine the role of Rap1 in regulating T cell proliferative responses, transgenic mice were generated that express RapV12 and B-Raf under a modified CD4 promoter (Sawada et al., Mol. Cell. Biol. 11:55.06-5515, 1991). This promoter directs expression in both CD4⁺ and CD8⁺ T cells. The RapV12 was linked to an N-terminal myc-epitope tag, to allow for discrimination between endogenous and ectopic Rap. In these mice myc-RapV12 is expressed at lower levels than endogenous Rap1. Therefore, it is likely that any consequence of RapV12 expression was not an artifact of overexpression (FIG. 2a).

B-Raf-expressing animals were also produced using the same CD4 promoter. In the thymus and lymph nodes of B-Raf transgenic mice a 96 kDa band, representing B-Raf protein, is seen by Western blot, in contrast to wild type animals which showed no expression of B-Raf (FIG. 2b). Similarly, immunohistochemical staining of the spleen and lymph node with B-Raf antibody showed no expression of B-Raf in these organs of a wild type animal. However, in the B-Raf transgenic animal, B-Raf expression was seen in all these lymphoid organs (FIG. 2c). B-Raf expression in the thymus was examined by FACS analysis. Intracellular B-Raf staining was identified in double negative (CD4/CD8^{-/-}), double positive (^{+/+}), CD4⁺/CD8⁻, and CD4⁻/CD8⁺ T cells from the B-Raf-transgenic, but not in the wild type mice (FIG. 2d).

To examine if introduction of the transgenes (B-Raf and RapV 12) had an effect on T cell development, we examined CD4 and CD8 expression on thymocytes. No differences were seen in CD4 and CD8 expression between the thymocytes isolated form wild type, B-Raf, RapV 12 and B-Raf×RapV 12 animals.

### Example 4

### Transgenic Expression Of B-Raf Increased ERK Activation In Response To CD3-ligation

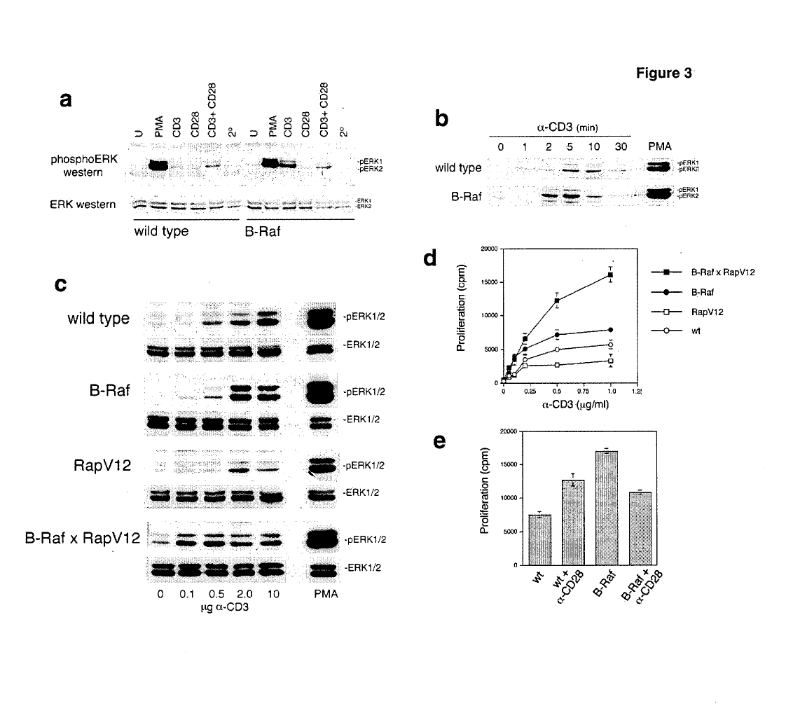

Rap1 activation could serve to limit ERK activation following TCR engagement by blocking Raf-1 action (Carey et al., Mol. Cell. Biol. 20:8409-8419, 2000). Expression of B-Raf in T cells is predicted to convert Rap1 into an activator of ERKs under conditions where Rap1 is activated. To test this hypothesis, the activation of ERKs, in T cells isolated from wild type and B-Raf transgenic animals was compared by Western blot, using anti-phospho-ERK antibodies (FIG. 3a). In both spleen and lymph node T cells, the activity of ERK1 and ERK2 was greatly increased in the B-Raf-expressing cells following CD3 ligation alone.

The effect of B-Raf on the ability of co-stimulation via CD28 ligation to augment ERK activation was examined. In wild type cells, co-stimulation enhanced the magnitude (FIG. 3a) and duration (FIG. 3b) of ERK activation. Interestingly, in the B-Raf-expressing cells, co-ligation of CD3 and CD28 did not lead to enhanced ERK activation but inhibited ERK activation below the levels seen by CD3 ligation alone (FIG. 3a). The levels of ERK activation in both the wild type and the B-Raf-expressing cells following co-stimulation were similar, suggesting that the increased ERK activation in B-Raf expressing cells following CD3 ligation alone could be inhibited following CD28 co-stimulation. This supports a model where CD28 negatively regulates Rap1 activity in both wild type and B-Raf cells. Since CD28 stimulation selectively inhibits Rap1 but not Ras (Carey et al., Mol. Cell. Biol. 20:8409-8419, 2000), the augmentation of CD3-induced ERK activation in the B-Raf-expressing T cells was likely mediated through Rap 1, not Ras.

### Example 5

### Transgenic Expression Of RapV12 And B-Raf Regulate ERK Activation Following TCR Ligation

To further investigate the ability of Rap1 to regulate ERK activation in primary T cells, mice transgenic for both B-Raf and RapV12 were generated by crossing the B-Raf-transgenic mice with the RapV12-transgenic mice. T cells were isolated from the four mouse lines (wild type, B-Raf, RapV12 and B-Raf×RapV12) and were stimulated with increasing concentrations of anti-CD3 antibody. ERK activation increased in a dose dependent manner in wild type T cells stimulated with anti-CD3 antibody (FIG. 3c, wild type) and this activation was enhanced in the B-Raf-expressing T cells (FIG. 3c, B-Raf). In the absence of B-Raf, the expression of RapV12 in T cells inhibited ERK activation at all concentrations of anti-CD3 antibody examined (FIG. 3c, RapV12). T cells isolated from the B-Raf×RapV12 mice showed constitutive activation of ERKs across all concentrations of anti-CD3 antibody (FIG. 3c, B-Raf×RapV12). These data show that RapV12 interfered with ERK activation following CD3 ligation, and that when co-expressed with B-Raf, RapV12 constitutively activated ERKs. Furthermore, B-Raf expression augmented ERK signaling in the absence of ectopic RapV12 expression, presumably via CD3-activated Rap 1.

### Example 6

### Transgenic Expression Of RapV12 And B-Raf Affect The Proliferation Of T Cells

To evaluate the functional consequences of B-Raf and RapV 12 in T cells, splenocytes were tested for rates of proliferation following CD3 stimulation (FIG. 3d). B-Raf-expressing T cells showed an increased proliferation rate following CD3 stimulation, compared to wild type cells, which paralleled the increase in ERK activity compared to wild type T cells. Expression of RapV12 in T cells significantly reduced the rate of proliferation of these cells compared to cells from the wild type mouse. Co-expression of B-Raf with RapV12 enhanced the proliferation rate of these cells, not only over that of wild type cells but also B-Raf-expressing cells.

### Example 7

### CD28 Co-ligation

Proliferation assays were performed using splenocytes isolated from either wild type or B-Raf-expressing animals stimulated with anti-CD3 antibody (0.25 μg/ml) in the presence and absence of agonist anti-CD28 antibody (FIG. 3e). As expected, wild type T cells proliferated at an increased rate in the presence of anti-CD28 antibody. Interestingly, in the presence of anti-CD28 antibody, the proliferation rate of B-Raf-expressing T cells was reduced from the level seen following CD3 stimulation alone to a level similar to that seen in the wild type T cells stimulated with anti-CD3 alone. These data are consistent with previous data that CD28 inhibited Rap1 activation of B-Raf in the B-Raf-expressing T cells and suggest that the increased proliferation of the B-Raf-expressing T cells was due to the increased signaling from Rap1 to ERKs via B-Raf.

### Example 8

### Rap1 Is Constitutively Active In Anergic T Cells

To investigate the role of Rap1 in anergy, the B-Raf transgenic mice were crossed with the AD10 TCR transgenic mice (AD10) and with the AND TCR transgenic mice, to generate mice that expressed both the transgenic TCR and B-Raf (AD10×B-Raf) or (AND×B-Raf) (Kaye et al., J. Immunol. 148:3342-3353, 1992).

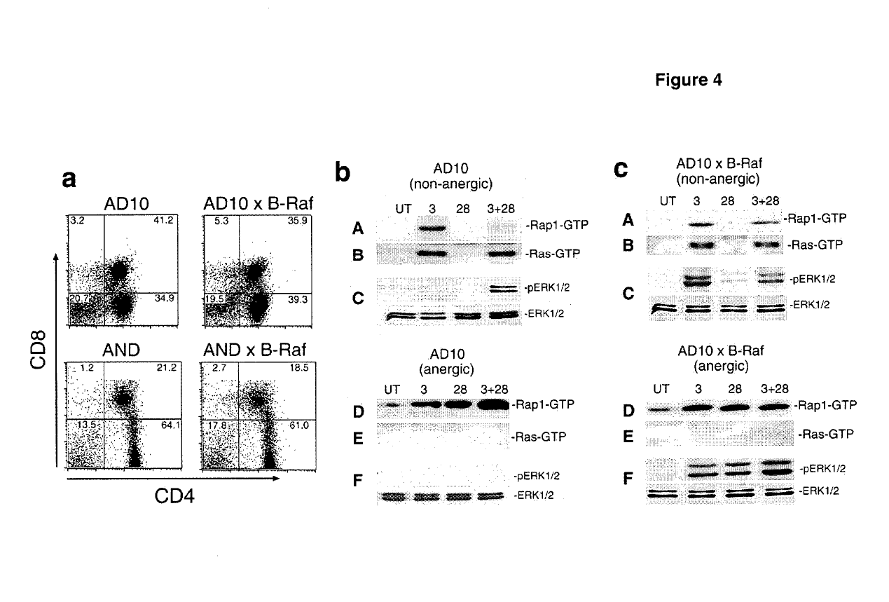

Thymocyte development was examined in the AD10 and AD10×B-Raf animals by examining CD4 and CD8 expression. No differences were seen in positive selection of T cells expressing the TCR transgenes within the CD4 compartment (FIG. 4a). Similarly, there were no differences in thymocyte development when B-Raf-expressing mice were crossed with the AND TCR transgenic mice (FIG. 4a).

Previous reports have shown that Rap1 is constitutively active in anergic T cells (Boussiotis et al., Science 278:124-128, 1997). Moreover, constitutive Rap1 activation has been proposed to be responsible for the defect in IL-2 production in anergy. First, Rap1 and ERK activation was examined in anergic and non-anergic T cells isolated from AD10 and AD10×B-Raf animals. When non-anergic AD10 T cells were re-stimulated with anti-CD3 antibody, both Rap1 and Ras were activated, and there was a corresponding moderate activation of ERKs (FIG. 4b, A, B, C). Under conditions of co-stimulation, Rap1 activation was reduced and ERK activation was enhanced although Ras activation remained the same (FIG. 4b, A, B, C). As shown previously (Boussiotis et al., Science 278:124-128, 1997), Rap1 was constitutively activated in anergic AD10 T cells (FIG. 4b, D). Interestingly, Rap1 was further activated upon re-stimulation of these cells, suggesting that Rap 1 was not maximally activated under basal conditions (FIG. 4b, D). Surprisingly, crosslinking with anti-CD28 antibodies also further activated Rap1 in anergic T cells, in contrast to non-anergic T cells. As previously reported, neither Ras nor ERKs were activated by anti-CD3 and/or anti-CD28 antibodies in anergic AD10 T cells (FIG. 4b, E, F) (Fields et al., Science 271:1276-1278, 1996; Li et al., Science 271:1272-1276, 1996).

Rap1 was activated in AD10×B-Raf T cells following re-stimulation with anti-CD3 antibody and its activation was modestly reduced under conditions of co-stimulation (FIG. 4c, A), whereas Ras was activated to similar degrees under both conditions (FIG. 4c, B). In contrast to non-anergic AD10 T cells, non-anergic AD10×B-Raf T cells showed a robust activation of ERKs under all conditions where Rap1 was activated (FIG. 4c, C).

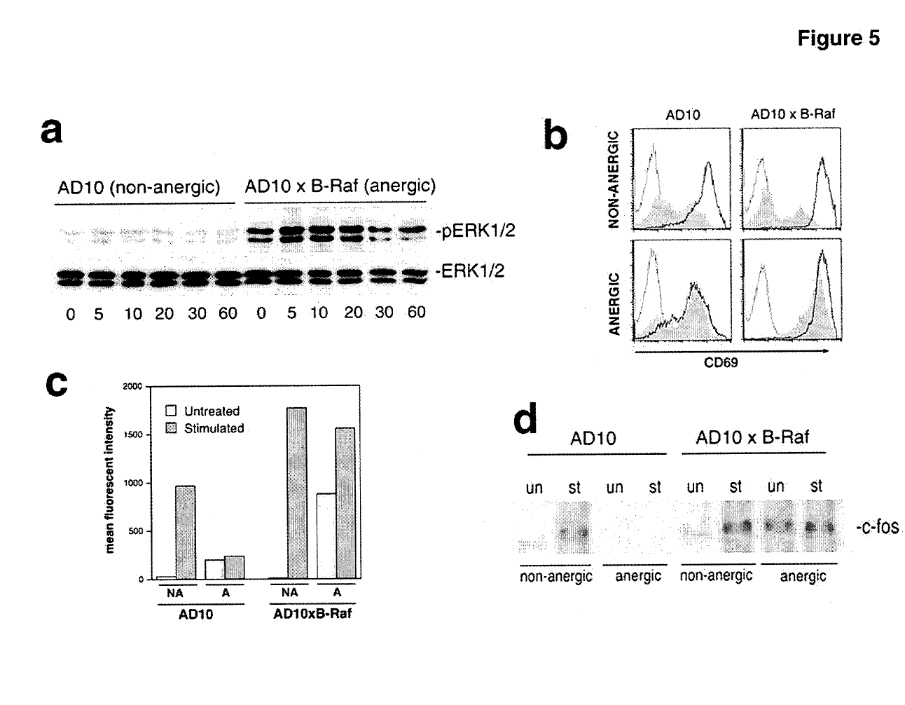

Similar to anergic AD10 T cells, Rap1 was constitutively activated in anergic AD10×B-Raf T cells but could be further activated by re-stimulation with both anti-CD3 and anti-CD28 antibodies (FIG. 4c, D). This activation of Rap1 in anergic AD10×B-Raf T cells was associated with the activation of ERKs (FIG. 4c, F), in contrast to anergic AD10 T cells. The enhanced ERK activity was not due to Ras activation of B-Raf since Ras was not activated in these anergic cells (FIG. 4c, E). These data provide strong evidence that Rap1 is signaling through B-Raf to activate ERKs. The enhancement of ERK activation during anergy was significant and was maintained for up to one hour after stimulation (FIG. 5a). One of the consequences of this enhanced ERK activation was the induction of genes that represent downstream targets of ERKs.

### Example 9

### ERK Activation In Anergic T Cells Can Activate Downstream Effectors

Up regulation of the early T cell activation marker, CD69, is both Ras (D'Ambrosio et al., Eur. J. Immunol. 24:616-620, 1994; Swat et al., Eur. J Immunol. 23:739-746, 1993) and ERK (Dumont et al., J. Immunol. 160:2579-2589, 1998) dependent. To verify that the elevated ERK expression seen in unstimulated and stimulated anergic cells is functional, CD69 expression was measured in restimulated anergic and non-anergic cells. As shown in FIG. 5b and 5c, in non-anergic cells, CD69 expression was enhanced following restimulation, as expected (D'Ambrosio et al., Eur. J Immunol. 24:616-620, 1994; Swat et al., Eur. J. Immunol. 23:739-746, 1993). B-Raf expression had no effect on this induction in non-anergic cells. In contrast, the expression of CD69 in anergic AD10×B-Raf animals was significantly higher than that seen in anergic AD10 animals, demonstrating that the elevated ERK activity seen during the induction of anergy was competent to couple to downstream events.

Another target for ERK in lymphocytes is the transcription factor c-fos (Mondino et al., J. Immunol. 157:2048-2057, 1996). Fos transcription is dependent on the ERK-dependent phosphorylation of the transcription factor Elk-1, a component of the ternary complex that binds to the serum response element within the c-fos promoter (Janknecht et al., EMBO J. 12:5097-5104, 1993). As shown in FIG. 5d, c-fos protein levels were increased following co-stimulation of non-anergic T cell blasts (from both AD10 and AD10×B-Raf animals). As expected, this was significantly decreased in anergic AD10 T cell blasts. In contrast, in anergic AD10×B-Raf blasts, both basal levels of c-fos protein and stimulated levels of c-fos protein were much higher than in AD10 wild type cells. This increase correlated with the increase in ERKs seen in these animals and suggests that increased ERK activity detected in anergic AD10-B-Baf cells was coupled to downstream effectors.

### Example 10

### Activation Of ERKs Is Not Sufficient To Rescue Anergy

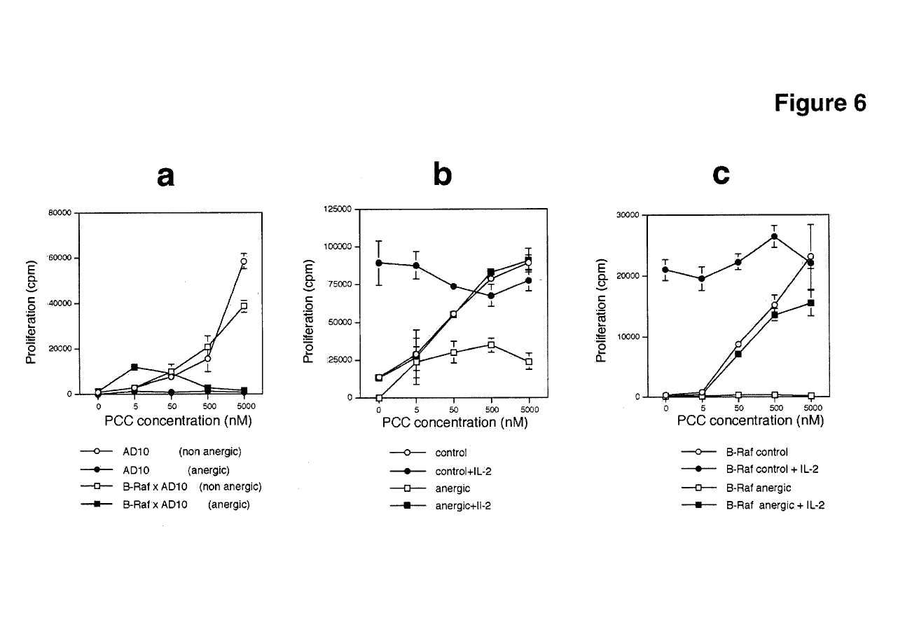

It is well established that anergic T cells neither proliferate nor make IL-2 in response to subsequent co-stimulation (Jenkins et al., J. Immunol. 144:16-22, 1990; Kang et al., Science 257:1134-1138, 1992). To examine whether Rap1's activation of ERKs via B-Raf could overcome this functional block, proliferation was measured in AD10 and AD10×B-Raf T cells. All non-anergic T cells (AD10 and A10×B-Raf) proliferated in response to antigenic signals (FIG. 6a) and as expected, AD10 anergic T cells did not proliferate. Surprisingly, anergic AD10×B-Raf T cells also did not proliferate (FIG. 6a). The addition of exogenous IL-2 rescued the proliferation of extracellular signal-regulated kinase both the AD10 and AD10×B-Raf anergic cells (FIGS. 6b and 6c), demonstrating that the inability of AD10×B-Raf T cells to proliferate was due to an inhibition of IL-2 production in these cells. These data suggest that ERK activation was not sufficient to rescue the proliferative defect of anergic T cells.

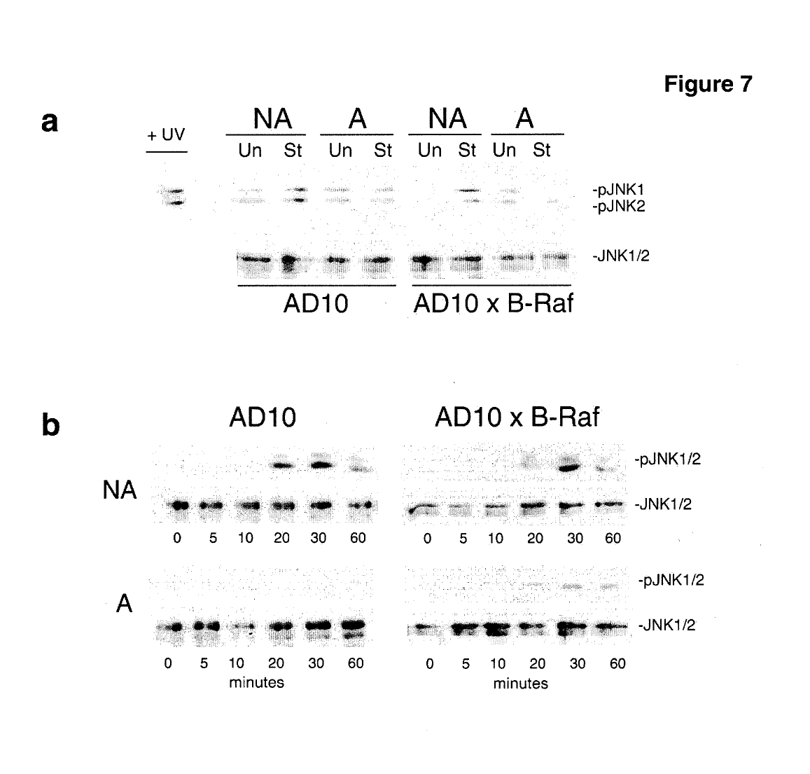

It has been suggested that defects in other signaling pathways might contribute to the maintenance of anergy. In particular the stress activated protein kinases [SAP kinases, or c-Jun N-terminal kinases (JNKs)] have been implicated in T cell activation and anergy (Mondino et al., J. Immunol. 157:2048-2057, 1996; Su et al., Cell 77:727-736, 1994; Werlen et al, EMBO J. 17:3101-3111, 1998. Examination of the phosphorylation state of JNKs revealed a significant loss of JNK activation following stimulation of both anergic AD10 and AD10×B-Raf lymphocytes (FIG. 7a). The inhibition of JNK activation following stimulation of anergic cells was seen at all time points examined (5-60 minutes) in both AD10 and AD10×B-Raf cells (FIG. 7b). Importantly, in the AD10×B-Raf cells, JNK activation was dramatically reduced following anergy induction, with only a residual JNK activation remained at later time points (FIG. 7b).

As demonstrated herein, one of the regulators of ERK activation in T cells is Rap1. Activation of Rap1 blocks Ras-dependent signaling to ERKs in multiple cell types, including fibroblasts and ovarian cells (Barberis et al., J. Biol. Chem. 275:36532-36540, 2000; Okada et al., EMBO J. 17:2554-2565, 1998). In T cells, Rap1 activation following TCR/CD3 ligation also serves to limit signals from Ras to ERKs (Carey et al., Mol. Cell. Biol. 20:8409-8419, 2000; Czyzyk et al., Mol. Cell. Biol. 20:8740-8747, 2000; Xing et al., Mol. Cell. Biol. 20:7363-7377, 2000). However, in B-Raf-expressing cells, Rap1 has the opposite action, to activate ERKs via B-Raf and the B-Raf effector, MEK. In cells that endogenously express both B-Raf and Raf-1, the positive action of Rap1 on B-Raf is dominant over the inhibitory actions of Rap1 on Raf-1, and the net effect of Rap1 activation is an increase in ERK activity (Chen et al., Cancer Res. 59:213-218, 1999; Schmitt et al., J. Biol. Chem. 275:25342-25350, 2000; Wan et al., J. Biol. Chem. 273:14533-14537, 1998.

As shown herein, endogenous T cells do not express B-Raf and that ectopic expression of B-Raf in both Jurkat cells and primary T cells converted Rap1 from an inhibitor to an activator of ERKs. Moreover, B-Raf expression converted constitutively activated Rap1 from an inhibitor of proliferation to an activator of proliferation.

One of the best-studied co-stimulatory signals provided by APCs is via the co-stimulatory receptor CD28 (Azuma et al., J. Ex. Med. 175:353-360 (1992). Co-ligation of CD28 has been shown to result in the inhibition of Rap1's activation by CD3, eliminating Rap1's ability to block Ras-dependent signals to ERKs. This action of CD28 to enhance ERK activation occurs in the absence of any direct action of CD28 on Ras itself (Carey et al., Mol. Cell. Biol 20:8409-8419 (2000). This model is supported by data showing that while CD28 co-stimulation of wild type T cells increased proliferation, CD28 co-stimulation of B-Raf-expressing T inhibited proliferation to levels seen with wild type cells stimulated with CD3 alone. These data are consistent with a model that CD28 limits Rap1 activation both in the B-Raf-expressing and wild type T cells.

Stimulation of the TCR/CD3 receptor complex in the absence of CD28 co-stimulatory signals produces a state of T cell unresponsiveness, or anergy, that is characterized by both diminished Ras (Boussiotis et al., Science 278:124-128, 1997; Fields et al., Science 271:1276-1278, 1996) and ERK activation (DeSilva et al., J. Exp. Med. 183:2017-2023, 1996; Li et al., Science 271:1272-1276, 1996) as well as constitutive activation of Rap1 (Boussiotis et al., Science 278:124-128, 1997). Constitutive activation of Rap1 has been proposed to account, in part, for both the diminished ERK activation and the lack of proliferative response following subsequent co-stimulation of anergic cells (Boussiotis et al., Science 278:124-128, 1997). Using an AD10 TCR transgenic mouse model, it is demonstrated herein that anergy can be induced in vitro and that this is associated with constitutive activation of Rap1. Interestingly, although Rap1 was activated to a limited degree in unstimulated anergic cells, it could be activated to much higher levels following stimulation via CD3, suggesting that Rap1 activation is not maximally induced during the induction of anergy in vitro. Under all conditions of anti-CD3 and/or anti-CD28 antibody stimulation of anergic AD10 cells, no activation of ERKs was seen. Surprisingly, CD28 co-stimulation did not reduce Rap1 activity in anergic cells as it had in non-anergic cells, but further activated Rap 1, suggesting that the induction of anergy had additional consequences on signals downstream of CD28. This inability of CD28 to down-regulate Rap1 in anergic cells may contribute to the lack of ERK activation seen in AD10 anergic cells following subsequent co-stimulation.

The introduction of B-Raf into T cells had a profound effect on the activation of ERKs in the anergic cell. B-Raf is an effector of both Ras and Rap. However, without being bound by theory, in anergic T cells it is likely that Rap1 activated B-Raf, since Ras activation was blocked in these cells. Under all conditions where Rap1 was activated, ERK was also strongly activated. This demonstrates that Rap1-mediated ERK activation occurred via B-Raf in AD10×B-Raf T cells. This elevated activation of ERKs was functional and resulted in elevated levels of membrane-associated CD69 and the transcription factor c-fos.

c-Fos has also been proposed as a target in anergic cells (Mondino et al., J. Immunol. 157:2048-2057, 1996). Furthermore, artificially elevating c-fos levels by ectopic expression confers resistance against anergy induction (Kawasaki et al., Int. Immunol. 11:1873-1880, 1999). The AD10×B-Raf cells displayed high level of constitutive c-fos expression in both anergic and non-anergic cells. Therefore, in contrast to previous studies, the AD10×B-Raf transgenic model permits examination of the consequence of elevation in endogenous c-fos protein. These studies demonstrate that elevated c-fos expression cannot, by itself, rescue the anergic phenotype.

It will be apparent that the precise details of the methods described may be varied or modified without departing from the spirit of the described invention. We claim all such modifications and variations that fall within the scope and spirit of the claims below.

1. A method of inducing T cell to proliferate in response to an antigen, comprising
introducing into the T cell a therapeutically effective amount of a nucleic acid encoding B-Raf, or a functional variant of B-Raf, operably linked to a promoter; thereby inducing the T cell to proliferate in response to the antigen.

2. The method of claim 1, wherein the T cell is in vivo.

3. The method of claim 1, wherein the T cell is in vitro.

4. The method of claim 1, further comprising contacting the T cell with the antigen.

5. The method of claim 1, wherein the T cell expresses CD4.

6. The method of claim 1, further comprising cross-linking CD28 on the T cell.

7. The method of claim 1, wherein expression of B-Raf activates extracellular signal-regulated kinase (ERK).

8. The method of claim 1, further comprising administering an antibody that specifically binds CD3.

9. The method of claim 1, wherein the antigen is expressed on a cell of a tumor.

10. A method of inducing an T cell to proliferate in response to an antigen, comprising
providing the T cell with a therapeutically effective amount of a composition that inhibits the expression or activity of Rap-1; thereby inducing the T cell to proliferate in response to the antigen.

11. The method of claim 10, wherein the composition that inhibits the expression or activity of Rap-1 is a nucleic acid encoding B-Raf, operably linked to a promoter.

12. The method of claim 10, wherein the T cell is in vivo.

13. The method of claim 10, wherein the T cell is in vitro.

14. The method of claim 10, further comprising contacting the T cell with an antigen.

15. The method of claim 10, wherein the T cell expresses CD4.

16. The method of claim 10, further comprising administering a therapeutically effective amount of IL-2, wherein the composition activates extracellular signal-regulated kinase (ERK).

17. The method of claim 16, wherein the composition is an antisense mRNA or a ribozyme specifically targeted to Rap-1.

18. The method of claim 10, wherein the antigen is a tumor antigen.

19. A method of inducing a response against an antigen in a subject, comprising administering a therapeutically effective amount of a nucleic acid sequence comprising a promoter operably linked to a nucleic acid sequence encoding B-Raf, thereby inducing the immune response against the antigen.

20. The method of claim 19, wherein the subject has a tumor.

21. The method of claim 19, wherein the antigen is a tumor antigen.

22. The method of claim 19, wherein the subject is a mammalian subject.

23. The method of claim 22, wherein the mammalian subject is a human subject.

24. The method of claim 19, wherein the promoter is promoter that is active in CD4+ T cells.

25. The method of claim 19, further comprising administering a therapeutically effective amount of the antigen or a nucleic acid encoding the antigen to the subject.

26. The method of claim 25, wherein the subject has a tumor.

27. The method of claim 25, wherein the antigen is a tumor antigen.

28. The method of claim 27, wherein the tumor antigen is carcinoembyonic antigen, prostate specific antigen.

29. A method for screening for an agent that activates B-raf, comprising
contacting an anergized T cell with the agent; and
detecting proliferation of the T cell, thereby determining if the agent activates B-raf.

30. The method of claim 30, further comprising
contacting the T cell with an antigen of interest.

31. The method of claim 30, wherein the activation of B-raf is increased transcription of mRNA encoding B-Raf.

32. The method of claim 30, wherein the activation of B-raf is increased production of B-raf polypeptide.

33. A transgenic mouse, wherein a nucleated cell of the transgenic mouse comprises a transgene encoding a B-Raf polypeptide, wherein T cells isolated from the transgenic non-human animal have increased extracellular signal-regulated kinase activity as compared to a wild-type mouse.
